(19) United States
(12) Patent Application Publication
(10) Pub. No.: US 2022/0226384 A1
(43) Pub. Date: Jul. 21, 2022

(54) EXTRACELLULAR VESICLE COMPOSITIONS AND METHODS OF USE THEREOF

(71) Applicant: Evia Life Sciences Inc., Seattle, WA (US)

(72) Inventors: Kensuke Tashiro, Chiba (JP); Takahiro Ochiya, Tokyo (JP)

(21) Appl. No.: 17/579,482

(22) Filed: Jan. 19, 2022

Related U.S. Application Data
(60) Provisional application No. 63/138,969, filed on Jan. 19, 2021.
(60) Provisional application No. 63/212,987, filed on Jun. 21, 2021.

(51) Int. Cl. A61K 35/28; A61P 37/00

(52) U.S. Cl. CPC A61K 35/28; A61P 37/00

(57) ABSTRACT

Compositions and methods for promoting generation or regeneration of the lymphatic system in a subject are provided. Compositions and methods for the treatment of lymphedema are also provided. The composition can include extracellular vesicles and a pharmaceutically acceptable carrier, and is typically cell-free. In some embodiments, the extracellular vesicles are formed by a method including culturing MSCs to produce media conditioned with the extracellular vesicles, and optionally, but preferably separating the extracellular vesicles from the media conditioned by the MSCs. In some embodiments, the extracellular vesicles include or consist of exosomes, microvesicles or a combination thereof, and they may have a size of between about 20 nm and about 500 nm. In some embodiments, extracellular vesicles include CD9, CD63, or a combination thereof and/or one or more of miR-199a-3p, miR-145-5p, miR-143-3p, miR-377-3p, miR-100-3p, miR-29a-3p, miR-495-3p, miR-29c-3p, miR-658, miR-493-3p, miR-184, and miR-27a-3p.

### CROSS-REFERENCE TO RELATED APPLICATION

This application claims the benefit of and priority to U.S. Ser. No. 63/138,969 filed Jan. 19, 2021, and U.S. Ser. No. 63/212,987 filed Jun. 21, 2021, each of which is incorporated by referenced herein in its entirety.

### REFERENCE TO THE SEQUENCE LISTING

The Sequence Listing submitted as a text file named “EVIA_100_ST25” created on Jan. 18, 2022 and having a size of 15,353 bytes is hereby incorporated by reference pursuant to 37 C.F.R. § 1.52(e)(5).

### FIELD OF THE INVENTION

The field of the invention generally relates to cell-free compositions including extracellular vesicles and methods of use thereof.

### BACKGROUND OF THE INVENTION

Lymphedema is based on a chronic disorder of the lymphatic system and accumulation of interstitial protein-rich fluid in the limbs. Lymphedema patients demonstrate chronic inflammation, and disorders of sensory and motor systems. Generally, lymphedema can be divided into primary and secondary types depending on the etiology. Primary lymphedema results from anatomic or functional defects, whereas secondary lymphedema is due to mainly infection or surgical resection of the lymph node for cancer therapy. It is estimated that approximately 30% of patients who undergo breast cancer surgery may develop lymphedema and 6% of cases of sentinel navigation surgery in the breast progress to lymphedema (DiSipio et al., Lancet Once, 14, 500-515 (2013)). In gynecologic cancers, approximately 10 to 30% of patients may develop lymphedema, depending on the cancer type, age, surgical approach. Furthermore, postoperative radiation increases the proportion of lymphedema to over 35% (Beesley et al., Cancer, 109, 2607-2614 (2007), Tada et al., BMC Cancer, 9, 47 (2009)). Because lymphedema disturbs the quality of life and elevates risks of recurrent cellulitis and secondary malignancy, the urgent development of effective treatment options is an important clinical goal.

To date, the treatment options are mainly limited to physiotherapy and surgical treatments. These methods require proper compliance and lifelong care. As the conventional treatment option, compression therapy using bandages or stockings is still used all over the world. Compression therapy can increase the internal pressure of the lymphatic systems. Thus, interstitial fluid could go into the lymphatic systems. Ideally, patients are required to wear thick garments or stockings every day even in summer; however, for most of the patients, full compliance is hard to achieve. In recent years, with the development of techniques using microsurgery and supermicrosurgery, surgical interventions such as lymphatico-venous anastomosis (LVA) have been attempted worldwide, with consistently favorable results. The long-term outcome of lymphatic microsurgery is also reported to be favorable in 24 to 48 months (Pedro, et al., Microsurgery, 40(2):130-136 (2020), and Antonio, et al., Gland Surg., 9(2):539-544 (2020)). However, these techniques are not easy to perform and are therefore not widely used in conventional clinical settings. Furthermore, it is still controversial whether the effect of surgical interventions lasts for an extended period. Moreover, such interventions cannot always stop the progress of the disease in severe cases. Regeneration of the lymphatic system is a hopeful treatment for the disease. Recent reports indicate that transplantation of mesenchymal stem cells (MSCs), such as adipose-derived mesenchymal stem cells (ADSCs) or bone-marrow mesenchymal stem cells (BMMSCs), promotes tissue regeneration including the lymphatic system by paracrine factors secreted by MSCs (Maertens et al., PLoS One, September 15;9(9):e106976 (2014), Shimizu et al., J Am Heart Assoc., August;1(4):e000877 (2012), Spees, et al., Stem Cell Res Ther., (7:125 (2016)). Diseases involving vascular and lymphatic systems such as ischemic heart failure, ischemic limb, DM foot necrosis, and lymphedema are possible disease indications. Transplantation of MSCs into damaged tissue has been shown to induce endothelial cell growth and enhance new blood vessel formation, with secreting paracrine factors as the predominant mechanism (Zhang, et al., J Transl Med. (13:49) (2015)).

As for inducing lymphangiogenesis, MSCs including ADSCs or BMMSCs have been shown to secrete many growth factors and cytokines that have effects on cells in their vicinity (Hwang, et al., Biomaterials, (32(19):4415-23 (2011)). For example, ADSCs can restore the lymphatic vascular network in a secondary mouse lymphedema model with increased collecting lymphatic vessels, mainly based on paracrine effects of ADSCs (Yoshida et al., Regen Med., 10(5):549-62 (2015)). In addition, it has been shown that BMMSCs play a role in lymphatic regeneration in a mouse tail lymphedema model (Conrad et al., Circulation, January 20;119(2):281-9 (2009)). In a clinical trial, BMMSCs were used for 10 post-mastectomy lymphedema patients, and the results indicate that BMMSC injection reduces arm volume as well as associated co-morbidities of pain (Maldonado et al., Cytotherapy, November;13(10):1249-55 (2011), Maldonado, et al., Cytotherapy, (13(10):1249-55 (2011)). ADSCs have been also used in both animal and clinical trials, showing their lymphangiogenic activity and therapeutic efficacy without serious adverse events in the six months follow-up period (Toyserkani et al., Stem Cells Transl Med., August;6(8):1666-1672 (2017), Hwang et al., Biomaterials, July;32(19):4415-23 (2011)). Potential risks of previous cancer spreading after treatment using MSCs are thought to be low because lymphedema treatment normally starts several years after initial treatment. However, MSC transplantation has drawbacks such as poor engraftment efficiency, potential tumor formation, unwanted immune responses, non-specific differentiation, and the difficulty of quality control before administration (Zhang, et al., Cell Pro 49:3-13 (2016)). Thus, there remains a need for alternatives to cell transplantation.

Thus, it is an object of the invention to provide alternative compositions and methods to mesenchymal stem cell transplantation.

It is a further object of the invention to provide compositions and methods of use thereof to promote generation or regeneration of the lymphatic system, treat lymphedema, and combinations thereof.

### SUMMARY OF THE INVENTION

Compositions and methods for promoting generation or regeneration of the lymphatic system in a subject are provided. Compositions and methods for the treatment of lymphedema are also provided. The compositions include extracellular vesicles, typically in a pharmaceutically acceptable carrier, and are typically cell-free. In some embodiments, the extracellular vesicles are formed by a method including culturing MSCs to produce media conditioned with the extracellular vesicles, and optionally, but preferably separating the extracellular vesicles from the conditioned media. Thus, in some embodiments, the composition does not include the media conditioned by the MSCs. The MSCs can be primary cells or a cell line. MSCs can be isolated or derived from, for example, bone barrow, placenta, umbilical cord blood, adipose tissue, adult muscle, corneal stroma, or the dental pulp of deciduous teeth. In a preferred embodiment, the composition includes extracellular vesicles formed by adipose-derived stem cells.

The extracellular vesicles can include or consist of ectosomes, microvesicles (MV), microparticles, exosomes, oncosomes, apoptotic bodies (AB), tunneling nanotubes (TNT), or a combination thereof. In some embodiments, the extracellular vesicles include or consist of exosomes, microvesicles or a combination thereof. The extracellular vesicles can include or consist of vesicles having a size of, for example, between about 20 nm and about 500 nm, or between about 20 nm and about 250 nm, or between about 20 nm and about 200 nm, or between about 20 nm and about 150 nm, or between about 20 nm and about 100 nm. In some embodiments, extracellular vesicles of the composition include CD9, CD63, or a combination thereof. In some embodiments, extracellular vesicles of the composition include one or more of miR-199a-3p, miR-145-5p, miR-143-3p, miR-377-3p, miR-100-3p, miR-29a-3p, miR-495-3p, miR-29c-3p, miR-658, miR-493-3p, miR-184, and miR-27a-3p.

In some embodiments the composition can increase the proliferation, migration, and/or tube formation of lymphatic endothelial cells, increase expression of one or more lymphatic markers (e.g., hyaluronan receptor-1 (LYVE-1), vascular endothelial growth factor receptor-3 (VEGFR-3), prospero homeobox 1 (Prox1), and/or podoplanin) in lymphatic endothelial cells, increase angiogenesis, increase lymphangiogeneisis, reduce inflammatory response, decrease fibrosis formation, enlarge circumference and/or induce formation of capillary vessels and/or lymphatic vessels, induce formation of vessels that express both vascular and lymphatic markers, increase drainage routes (e.g., for accumulated fluids), increase HIF1-alpha expression and/or activity (e.g., in lymphatic endothelial cells), reduce Prohibitin (PHB) expression and/or activity (e.g., in lymphatic endothelial cells), or a combination thereof in the subject.

The methods typically include administering a subject in need thereof a composition having an effective amount of extracellular vesicles formed by mesenchymal stem cells (MSCs) to, for example, increase generation of the lymphatic system or reduce one or more symptoms of lymphedema. The subject can have lymphedema or one or more symptoms thereof such as swelling of part or all of the arm(s) and/or leg(s), a feeling of heaviness or tightness, restricted range of motion, aching or discomfort, recurring infections, and fibrosis in one or both arms and/or legs. The subject can have a blockage in the lymphatic system, optionally wherein the blockage prevents lymph fluid from draining well, and wherein the fluid buildup leads to swelling.

In some methods, the composition is administered by local injection or infusion at or adjacent to a site of interest, for example in one or both arms and/or legs. The site of interest can be a site of lymphatic blockage and/or lymphedema or another site in need of lymphatic system generation or regeneration. The composition can be administered by any suitable means including, but not limited to, intramuscular, intraperitoneal, intravenous, subcutaneous, or subdermal injection or infusion.

### BRIEF DESCRIPTION OF THE DRAWINGS

FIG. 1 is a plot showing nanotracking analysis of the size distribution of extracellular vesicles (EV) isolated from conditioned media of adipose-derived stem cells.

FIGS. 2A-2C are bar graphs showing proliferation (2A, WST-8 assay), migration (2B, Boyden chamber assay), and tube length (2C, pixel length per field (×40 magnification, 5 random fields)) of lymphatic endothelial cells (LECs) treated with PBS, HEK 293-EVs, VEGF-C, or adipose-derived stem cells (ADSC)-EVs. For FIGS. 2A and 2B, values are means (SD) (n=6, triplicate) (*P<0.05). For FIG. 2C, values are means (SD) (n=6, duplicate) (*P<0.05).

FIGS. 3A-3H are bar graphs showing mRNA expression levels of LYVE-1 (3A, 3E), VEGFR-3 (3B, 3F), Prox1 (3C, 3G) and podoplanin (3D, 3H) in LECs 12 hrs. (3A-3D) or 24 hrs (3E-3H) after treatment with PBS, VEGF-C, or ADSC-EVs. The samples were analyzed by qRT-PCR to evaluate the expression of genes. The data were normalized based on GAPDH expression and shown as changes relative to PBS group. Values are means (SD) (n=3, triplicate) (*P<0.05 vs PBS). GAPDH indicates glyceraldehyde-3-phosphate dehydrogenase.

FIG. 4A is a schematic illustrating a splinted lymphedema model. After x-ray irradiation in the bilateral inguinal region at 10 Gy in a single dose twice prior to the surgery, mice were subjected to circumferential incision in the inguinal region. After the resection of inguinal lymph nodes, a 3-mm-wide silicone splint was placed in the inguinal wound and then fixed to the skin and underlying muscle to prevent wound contraction and desiccation. As a therapeutic intervention, injection of PBS or ADSC-EVs (˜40 μg) or HEK293-EVs (˜40 μg) as a thin layer to the whole leg area was performed in postoperative day 7 and 14. FIG. 4B is a line graph showing the ratio of hindlimb circumference change over 4 weeks after injections of PBS or ADSC-EVs. FIG. 4C is a bar graph showing quantitation of computed tomographic images of indocyanine green (ICG) lymphography used to assess lymphatic function of the hindlimb, after indocyanine green injection of both feet.

FIGS. 5A-5C are bar graphs showing histological analysis of LYVE-1(+) area (%) (5A), CD31(+) area (%) (5B), CD31(+)/LYVE-1(+) overlap area (%) (5C) per 0.25 mm2 area (four random areas per limb) following treatment with PBS, HEK 293-EVs, or ADSC-EVs. All data represent mean±s.d. with P<0.05 considered as significant.

FIGS. 6A-6B are bar graphs showing collagen 1(+) area (%) (6A) and pSMAD3(+) area (%) (6B) per 0.25 mm2 area (four random areas per limb) after treatments of PBS, HEK293-EVs, or ADSC-EVs. All data represent mean±s.d. with P<0.05 considered as significant.

FIG. 7 is a gene interaction network map illustrating the results of an Ingenuity Pathway Analysis (IPA) evaluating the implications of miRNA expression found to be altered by ADSC-EVs treatment. This map provides the top ranked network found associated with the role of ADSC-EVs in lymphatic endothelial cells.

### DETAILED DESCRIPTION OF THE INVENTION

### I. Definitions

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents. As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject. As used herein, “substantially changed” means a change of at least e.g. 5%, 10%, 15%, 20%, 25%, 30%, 35%, 40%, 45%, 50%, 75%, 100%, or more relative to a control.

As used herein, the term “purified,” “isolated,” and like terms relate to the isolation of a molecule or compound in a form that is substantially free (at least 60% free, preferably 75% free, and most preferably 90% free) from other components normally associated with the molecule or compound in a native environment.

As used herein, the term “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen.

As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the terms “inhibit” or “reduce” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” or “reduce” can also mean to hinder or restrain the synthesis, expression or function of a protein relative to a standard or control. This can include, but is not limited to, the complete ablation of the activity, response, condition, or disease. Inhibition may also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels.

Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### II. Compositions

In general, cell-based therapies have limitations such as uncontrolled differentiation, side effects, tumor formation, and incompatibility of allogenic use. On the contrary, regenerative therapy using extracellular vesicles (EVs) from MSCs have the possibility to overcome such disadvantages cell-based therapies have.

The conditioned medium which contains cultured MSCs secretion have also been reported to have lymphangiogenic effects and therapeutic potential (Takeda et al., Ann Plast Surg., June;74(6):728-36 (2015)). MSCs can secrete cytokines, chemokines, growth factors, and EVs (Katsuda et al., Proteomics, May;13(10-11) (2013)). These EVs are produced by a variety of cell types and may pay a role as intercellular transmitters of mRNA, microRNA, and proteins (Valadi et al., Nature Cell Biology volume 9, pages 654-659 (2007)). There is some evidence indicating that some of the regenerative properties previously credited to MSCs may be related to the secreted EVs (Lai et al., Stem Cell Res., May;4(3):214-22 (2010), Bruno et al., J Am Soc Nephrol., May;20(5):1053-67 (2009)). However, the experiments discussed in the Examples below investigate the therapeutic ability of EVs secreted from ADSCs using in vitro and in vivo experimental systems and demonstrate that ADSC-EVs can modulate the phenotype of lymphatic endothelial cells (LECs), which may contribute to several lymphatic pathophysiological processes. Results further demonstrate the therapeutic potential of ADSC-EVs in relief of lymphedema using a mouse hindlimb lymphedema model. Cell-free compositions including EVs and methods of use thereof are provided. The EVs can be part of a heterogeneous mixture of factors such as conditioned media, or a fraction isolated therefrom. In other embodiments, EVs, or one or more subtypes thereof, are isolated or otherwise collected from conditioned media. The EVs, or one or more subtypes thereof, can be suspended in a pharmaceutically acceptable composition, such as a carrier or matrix or depot, prior to administration to the subject.

A. Extracellular Vesicles

The disclosed compositions typically are or include extracellular vesicles derived from mesenchymal cells, or an isolated or fractionated subtype or subtypes thereof. Extracellular vesicles are lipid bilayer-delimited particles that are naturally released from a cell and, unlike a cell, cannot replicate. EVs range in diameter from near the size of the smallest physically possible unilamellar liposome (around 20-30 nanometers) to as large as 10 microns or more, although the vast majority of EVs are smaller than 200 nm. EVs secreted from MSCs play a role in MSC-mediated paracrine effects via transfer of miRNAs (Spees et al., Stem Cell Res Ther., 7:125 (2016)), and may have effects on wound healing (Zhang et al., J Transl Med., February 1;13:49 (2015)), skin rejuvenation (Kim et al., Biochem Biophys Res Commun., November 18;493(2):1102-1108 (2017)), angiogenesis (Gong et al., Oncotarget, July 11;8(28):45200-45212 (2017)), adjusting immunologic function (Zhao et al., Diabetes, February;67(2):235-247 (2018)), regeneration of damaged tissue (Lai et al., Stem Cell Res., May;4(3):214-22 (2010)) as well as relief neurological disorders (Katsuda et al., Proteomics, May;13(10-11) (2013)). Diverse EV subtypes have been proposed including ectosomes, microvesicles (MV), microparticles, exosomes, oncosomes, apoptotic bodies (AB), tunneling nanotubes (TNT), and more (Yáñez-Mó, et al., J Extracell Vesicles. 4: 27066 (2015) doi:10.3402/jev.v4.27066. PMC 4433489). These EV subtypes have been defined by various, often overlapping, definitions, based mostly on biogenesis (cell pathway, cell or tissue identity, condition of origin) (Théry, et al., J Extracell Vesicles. 7 (1): 1535750 (2018). doi:10.1080/20013078.2018.1535750). However, EV subtypes may also be defined by size, constituent molecules, function, or method of separation. According to minimal information from studies of extracellular vesicles 2018 (MISEV2018)12, EV is a collective term covering various subtypes of cell-released, membranous structures, called exosomes, microvesicles, microparticles, ectosomes, oncosomes, apoptotic bodies, and many other names (Clotilde, et al., J Extracell Vesicles, 7(1):1535750 (2018)). As discussed in Théry, et al., subtypes of EVs may be defined by:
a) physical characteristics of EVs, such as size (“small EVs” (sEVs) and “medium/large EVs” (m/lEVs), with ranges defined, for instance, respectively, <100 nm or <200 nm [small], or >200 nm [large and/or medium]) or density (low, middle, high, with each range defined);
b) biochemical composition (CD63+/CD81+−EVs, Annexin A5-stained EVs, etc.); or
c) descriptions of conditions or cell of origin (podocyte EVs, hypoxic EVs, large oncosomes, apoptotic bodies).

Thus, in some embodiments, the composition is or includes one or more EV subtypes defined according (a), (b), or (c) as discussed above.

In some embodiments, the vesicles are or include exosomes. Exosomes possess the surface proteins that promote endocytosis and they have the potential to deliver macromolecules. Also, if the exosomes are obtained from the same individual as they are delivered to, the exosomes will be immunotolerant. Due to the technical limitations, previous studies are not sufficient to conclude that exosomes have specific functions compared with other EVs (Clotilde, et al., J Extracell Vesicles, 7(1):1535750 (2018)).

Exosomes are vesicles with the size of 30-150 nm, often 40-100 nm, and are observed in most cell types. Exosomes are often similar to MVs with an important difference: instead of originating directly from the plasma membrane, they are generated by inward budding into multivesicular bodies (MVBs). The formation of exosomes includes three different stages: (1) the formation of endocytic vesicles from plasma membrane, (2) the inward budding of the endosomal vesicle membrane resulting in MVBs that consist of intraluminal vesicles (ILVs), and (3) the fusion of these MVBs with the plasma membrane, which releases the vesicular contents, known as exosomes.

Exosomes have a lipid bilayer with an average thickness of ˜5 nm (see e.g., Li, Theranostics, 7(3):789-804 (2017) doi: 10.7150/thno.18133).

The lipid components of exosomes include ceramide (sometimes used to differentiate exosomes from lysosomes), cholesterol, sphingolipids, and phosphoglycerides with long and saturated fatty-acyl chains. The outer surface of exosomes is typically rich in saccharide chains, such as mannose, polylactosamine, alpha-2,6 sialic acid, and N-linked glycans.

Many exosomes contain proteins such as platelet derived growth factor receptor, lactadherin, transmembrane proteins and lysosome associated membrane protein-2B, membrane transport and fusion proteins like annexins, flotillins, GTPases, heat shock proteins, tetraspanins, proteins involved in multivesicular body biogenesis, as well as lipid-related proteins and phospholipases. These characteristic proteins therefore serve as good biomarkers for the isolation and quantification of exosomes. Another key cargo that exosomes carry is nucleic acids including deoxynucleic acids (DNA), coding and non-coding ribonucleic acid (RNA) like messenger RNA (mRNA) and microRNA (miRNA).

In some embodiments, the vesicles include or are one or more alternative extracellular vesicles, such as ABs, MVs, TNTs, or others discussed herein or elsewhere.

ABs are heterogenous in size and originate from the plasma membrane. They can be released from all cell types and are about 1-5 μm in size.

MVs with the size of 20 nm-1 μm are formed due to blebbing with incorporation of cytosolic proteins. In contrast to ABs, the shape of MVs is homogenous. They originate from the plasma membrane and are observed in most cell types.

TNTs are thin (e.g., 50-700 nm) and up to 100 μm long actin containing tubes formed from the plasma membrane.

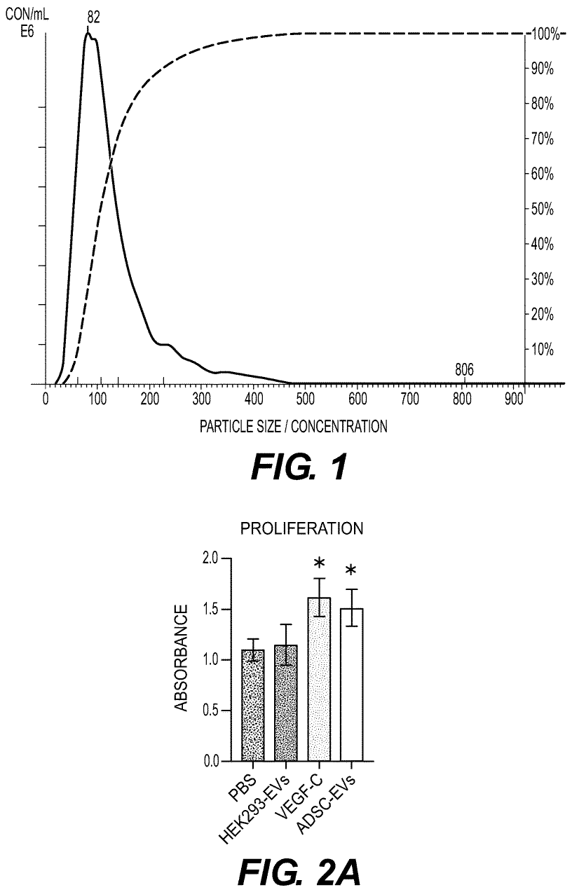

The Examples below show that EVs isolated from ADSC were physically homogeneous with a peak of 100 nm (FIG. 1). Thus, in some embodiments, the EVs are between about 20 nm and about 500 nm. In some embodiments, the EVs are between about 20 nm and about 250 nm or 200 nm or 150 nm or 100 nm.

The Examples below show that EVs isolated from ADSC include CD9 and CD63. Thus, in some embodiments, the EVs include CD9, CD63, or both.

The Examples below show that EVs isolated from ADSC include a number of miRNAs. In some embodiments, the EVs include one or more of the miRNA of Table 1. In some embodiments, the EVs include on or more of miR-199a-3p, miR-145-5p, miR-143-3p, miR-377-3p, miR-100-3p, miR-29a-3p, miR-495-3p, miR-29c-3p, miR-658, miR-493-3p, miR-184, and miR-27a-3p.

B. Methods of Making Extracellular Vesicles

1. Sources of Cells for Making Extracellular Vesicles

As used herein, EVs, including AB, MV, exosomes, and TNT typically refer to lipid vesicles formed by cells or tissue. They can be isolated from tissue, cells, and fluid directly from a subject, including cultured and uncultured tissue, cells, or fluids, and fluid derived or conditioned by cultured cells (e.g., conditioned media). For example, exosomes are present in physiological fluids such as plasma, lymph liquid, malignant pleural effusion, amniotic liquid, breast milk, semen, saliva and urine, and are secreted into the media of cultured cells.

The EVs of the disclosed compositions are typically formed from mesenchymal cells, preferably mesenchymal stem cells. Mesenchymal stem cells (MSCs) are multipotent adult stem cells that are present in multiple tissues, including umbilical cord, bone marrow and fat tissue. Mesenchymal stem cells can self-renew by dividing and can differentiate into multiple tissues including bone, cartilage, muscle and fat cells, and connective tissue. Thus, mesenchymal cells include, for example, adipocytes, chondrocytes, osteoblasts, myocytes and tendon, and MSCs are multipotent stem cells that can differentiate in one or more of these cell types.

In some embodiments, the EVs are formed by MSCs. MSCs can be derived from bone barrow or other non-marrow tissues, such as placenta, umbilical cord blood, adipose tissue, adult muscle, corneal stroma or the dental pulp of deciduous (baby) teeth.

In some embodiments, the MSCs are derived from adipose tissue. Adipose tissue-derived MSCs (AdMSCs or ADSC), in addition to being easier and safer to isolate than bone marrow-derived MSCs, can be obtained in larger quantities.

Methods of isolating extracellular vesicles from tissue, cells, and fluid directly from a subject, including cultured and uncultured tissue, cells, or fluids, and fluid derived or conditioned by cultured cells (e.g., conditioned media) are known in the art.

See, for example, Li, Themaostics, 7(3):789-804 (2017) doi: 10.7150/thno.18133, Ha, et al., Acta Pharmaceutica Sinica B, 6(4):287-296 (2016) doi: 10.1016/j.apsb.2016.02.001, Skotland, et al., Progress in Lipid Research, 66:30-41 (2017) doi: 10.1016/j.plipres.2017.03.001, Phinney and Pittenger, Stem Cells, 35:851-858 (2017) doi: 10.1002/stem.2575, each of which is specifically incorporated by reference, and describes isolating extracellular vesicles, particularly exosomes.

The EVs can be collected from primary cells or tissue or fluid. In some embodiments, the vesicles are isolated from cells, tissue, or fluid of the subject to be treated. An advantage of utilizing EVs that are isolated from natural sources includes avoidance of immunogenicity that can be associated with artificially produced lipid vesicles.

The EVs can also be collected from cell lines or tissue. Exemplary cells lines are commercially available and include those various sources including human bone-marrow, human umbilical cord, human embryonic tissue, and human adipose including those derived from lipoaspirate or dedifferentiated from mature adipocytes.

2. Methods of Collecting Extracellular Vesicles

Extracellular vesicles, including exosomes, can be isolated using differential centrifugation, flotation density gradient centrifugation, filtration, high performance liquid chromatography, and immunoaffinity-capture.

For example, one of the most common isolation techniques for isolating exosomes from cell culture is differential centrifugation, whereby large particles and cell debris in the culture medium are separated using centrifugal force between 200-100,000×g and the exosomes are separated from supernatant by the sedimenting exosomes at about 100,000×g. Purity can be improved, however, by centrifuging the samples using flotation density gradient centrifugation with sucrose or Optiprep. Tangential flow filtration combined with deuterium/sucrose-based density gradient ultracentrifugation was employed to isolate therapeutic exosomes for clinical trials.

In the experiments provided below, EVs were isolated from ADSCs. After incubation for two days, the medium was collected and centrifuged at 2,000 g for 15 min at room temperature. To thoroughly remove cellular debris, the supernatant was filtered with a 0.22-mm filter unit. Then, the cultured media (CM) was ultracentrifuged at 110,000 g (35,000 rpm) for 70 min at 4° C.

Ultrafiltration and high-performance liquid chromatography (HPLC) are additional methods of isolating EVs based on their size differences. EVs prepared by HPLC are highly purified.

Hydrostatic filtration dialysis has been used for isolating extracellular vesicles from urine.

Other common techniques for EV collection involve positive and/or negative selection using affinity-based methodology. Antibodies can be immobilized in different media conditions and combined with magnetic beads, chromatographic matrix, plates, and microfluidic devices for separation. For example, antibodies against exosome-associated antigens—such as cluster of differentiation (CD) molecules CD63, CD81, CD82, CD9, epithelial cell adhesion molecule (EpCAM), and Ras-related protein (Rab5)—can be used for affinity-based separation of exosomes. Non-exosomes vesicles that carry these or different antigens can also be isolated in a similar way.

Microfluidics-based devices have also been used to rapidly and efficiently isolate EVs such as exosomes, tapping on both the physical and biochemical properties of exosomes at microscales. In addition to size, density, and immunoaffinity, sorting mechanisms such as acoustic, electrophoretic and electromagnetic manipulations can be implemented.

Methods of characterizing EVs including exosomes are also known in the art. Exosomes can be characterized based on their size, protein content, and lipid content. Exosomes are sphere-shaped structures with sizes between 40-100 nm and are much smaller compared to other systems, such as a microvesicle, which has a size range from 100-500 nm. Several methods can be used to characterize EVs, including flow cytometry, nanoparticle tracking analysis, dynamic light scattering, western blot, mass spectrometry, and microscopy techniques. EVs can also be characterized and marked based on their protein compositions. For example, integrins and tetraspanins are two of the most abundant proteins found in exosomes. Other protein markers include TSG101, ALG-2 interacting protein X (ALIX), flotillin 1, and cell adhesion molecules. Similar to proteins, lipids are major components of EVs and can be utilized to characterize them.

C. Pharmaceutical Compositions

Pharmaceutical compositions including EVs are also provided. Pharmaceutical compositions can be administered parenterally (intramuscular (IM), intraperitoneal (IP), intravenous (IV), subcutaneous injection (SubQ), subdermal), transdermally (either passively or using iontophoresis or electroporation), or by any other suitable means, and can be formulated in dosage forms appropriate for each route of administration.

In some embodiments, the compositions are administered systemically, for example, by intravenous or intraperitoneal administration, in an amount effective for delivery of the compositions to targeted cells.

In preferred embodiments, the compositions are administered locally, for example, by injection directly into, or adjacent to, a site to be treated. For example, in some embodiments such as for the treatment of lymphedema, the compositions are injected or otherwise administered directly to the lymphedemic area or the area adjacent thereto (e.g., in the arms or legs).

Typically, local injection causes an increased localized concentration of the compositions which is greater than that which can be achieved by systemic administration.

In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems: Fundamentals and Techniques (Chichester, England: Ellis Horwood Ltd., 1988 ISBN-10: 0895735806), which can effect a sustained release of the material to the immediate area of the implant.

The EV compositions can be provided to the cell either directly, such as by contacting it with the cell, or indirectly, such as through the action of any biological process. For example, the vesicles can be formulated in a physiologically acceptable carrier, and injected into a tissue or fluid surrounding the cell.

Exemplary dosage for in vivo methods are discussed in the experiments below. As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally, for local injection or infusion, dosage may be lower. Generally, the total amount of the active agent administered to an individual using the disclosed vesicles can be less than the amount of unassociated active agent that must be administered for the same desired or intended effect and/or may exhibit reduced toxicity.

In a preferred embodiment the compositions are administered in an aqueous solution, by parenteral injection such as intramuscular, intraperitoneal, intravenous, subcutaneous, subdermal, etc.

The formulation can be in the form of a suspension or emulsion. In general, pharmaceutical compositions are provided including effective amounts of one or more active agents optionally include pharmaceutically acceptable diluents, preservatives, solubilizers, emulsifiers, adjuvants and/or carriers. Such compositions can include diluents, sterile water, buffered saline of various buffer content (e.g., Tris-HCl, acetate, phosphate) at various pHs and ionic strengths; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

Transdermal formulations may also be prepared. These will typically be ointments, lotions, sprays, or patches, all of which can be prepared using standard technology. Transdermal formulations can include penetration enhancers. Chemical enhancers and physical methods including electroporation and microneedles can work in conjunction with this method.

### III. Methods of Use

Methods of using the disclosed compositions are also provided. The experiments below illustrate that EVs from ADSCs promoted proliferation, migration, and tube formation activities, and upregulated gene expression of lymphatic markers in lymphatic endothelial cells. ADSC-EVs increased both LYVE-1 positive lymphatic endothelial cells and CD-31 positive vascular endothelial cells of injected limbs in vivo, and suppressed immunologic activities. These alterations induced by ADSC-EVs resulted in relief the condition of lymphedema.

Thus, in some embodiments, the disclosed compositions are administered to a subject in need thereof in an effective amount. In some embodiments, the amount is effective to increase the proliferation, migration, and/or tube formation of lymphatic endothelial cells, increase expression of one or more lymphatic makers (e.g., hyaluronan receptor-1 (LYVE-1), vascular endothelial growth factor receptor-3 (VEGFR-3), prospero homeobox 1 (Prox1), and/or podoplanin) in lymphatic endothelial cells, increase angiogenesis, increase lymphangiogeneisis, reduce inflammatory response, decrease fibrosis formation, enlarging circumference and/or inducing formation of capillary vessels and/or lymphatic vessels, induce formation of vessels that express both vascular and lymphatic markers, increase drainage routes (e.g., for accumulated fluids), increase HIF1-alpha expression and/or activity (e.g., in lymphatic endothelial cells), reduce Prohibitin (PHB) expression and/or activity (e.g., in lymphatic endothelial cells), or a combination thereof in a subject.

The disclosed compositions and methods are particularly useful for treating a subject having lymphedema, and/or symptoms associated therewith. Thus, in some embodiments, EVs or a subtype or subtypes thereof or a composition thereof, are administered to a subject in need thereof in an effective amount to treat lymphedema, or one or more symptoms associated therewith.

Lymphedema refers to swelling that generally occurs in one or both arms or legs. Lymphedema is generally caused by blockage the lymphatic system. The blockage prevents lymph fluid from draining well, and the fluid buildup leads to swelling.

Lymphedema signs and symptoms, which typically occur in the affected arm(s) and/or leg(s), include: swelling of part or all of the arm(s) and/or leg(s), including fingers or toes, a feeling of heaviness or tightness, restricted range of motion, aching or discomfort, recurring infections, hardening and thickening of the skin (fibrosis).

Draining excess lymphatic fluid is one of the main purposes of lymphedema treatment. To promote remaining drainage function, traditional compression care or microsurgical lymphatic system reconstruction has been performed for lymphedema management (Tashiro, et al., J Plast Reconstr Aesthet Surg., 69(3):368-75 (2016)). Recent advancement of surgical approach to lymphedema has been attempted to create lympho-venous shunts or healthy lymph nodes transplantation from other sites. Thus, in some embodiments, the disclosed methods are combined with one or more of these or other conventional methods of treating lymphedema.

In the studies below, the effects of ADSC-EVs treatment were investigated both in vitro and in vivo situations, and results indicate that inducing both angiogenesis and lymphangiogenesis may lead to the establishment of new connection between capillary vessels and lymphatic vessels. The existence of the route between capillary system and lymphatic system may work lifelong as drainage route of excess lymphatic fluid in affected tissues as a treatment option.

Lymphedema can be caused by the removal of, or damage to, lymph nodes as a part of cancer treatment. Thus, in some embodiments, the subject also has cancer. In some embodiments, the subject does not have cancer.

In some embodiments, the EVs are administered as part of a heterogeneous mixture of factors (e.g., conditioned media, or a fraction isolated therefrom). In some embodiments, EVs or more of more subtypes thereof are isolated or otherwise collected from conditioned media. The EVs or one or more subtypes thereof can be suspended in pharmaceutically acceptable composition, such as a carrier or matrix or depot, prior to administration to the subject.

EVs may possess the versatility and capacity to interact with multiple cell types immediately and remote areas to regulate cellular responses (Zhang et al., Cell Prolif., 49:3-13 (2016)). Thus, although regional or local administration to the site of interest (e.g., the site of lymphedema) or a site adjacent thereto is preferred, systemic administration is also contemplated. Furthermore, although lymphatic endothelial cells are a preferred target, the EVs may also affect other cells in the region of administration that effect the treatment outcome.

The frequency of administration of a method of treatment can be, for example, one, two, three, four or more times daily, weekly, every two weeks, or monthly. In some embodiments, the composition is administered to a subject once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the frequency of administration is once, twice or three times weekly, or is once, twice or three times every two weeks, or is once, twice or three times every four weeks. In some embodiments, the composition is administered to a subject 1-3 times, preferably 2 times, a week.

In some embodiments, the effect of the disclosed compositions and methods on a subject is compared to a control. For example, the effect of the composition on a particular symptom, pharmacologic, or physiologic indicator (including those mentioned above and elsewhere herein) can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects).

In some embodiments, the effect of the treatment is compared to a conventional treatment that is known in the art, such as one of those discussed herein.

### IV. Kits

Dosage units including the disclosed compositions, for example, in a pharmaceutically acceptable carrier for shipping and storage and/or administration are also disclosed. Components of the kit may be packaged individually and can be sterile. In some embodiments, a pharmaceutically acceptable carrier containing an effective amount of the composition is shipped and stored in a sterile vial. The sterile vial may contain enough composition for one or more doses. The composition may be shipped and stored in a volume suitable for administration, or may be provided in a concentration that is diluted prior to administration. In another embodiment, a pharmaceutically acceptable carrier containing drug can be shipped and stored in a syringe.

Kits containing syringes of various capacities or vessels with deformable sides (e.g., plastic vessels or plastic-sided vessels) that can be squeezed to force a liquid composition out of an orifice are provided. The size and design of the syringe will depend on the route of administration. Any of the kits can include instructions for use.

The disclosed compositions and methods can be further understood through the following numbered paragraphs.

1. A method of promoting generation or regeneration of the lymphatic system in a subject comprising administering the subject a composition comprising an effective amount of extracellular vesicles formed by mesenchymal stem cells (MSCs) to increase generation of the lymphatic system. 2. A method of treating a subject for lymphedema comprising administering the subject an effective amount of a composition comprising extracellular vesicles formed by mesenchymal stem cells to increase generation of the lymphatic system.

3. The method of paragraphs 1 or 2, wherein the composition comprises a pharmaceutically acceptable carrier.

4. The method of any one of paragraphs 1-3, wherein the composition is cell-free.

5. The method of any one of paragraphs 1-4, wherein the extracellular vesicles are formed by a method comprising culturing MSCs to produce media conditioned with the extracellular vesicles.

6. The method of paragraph 5, wherein the method further comprises separating extracellular vesicles from the media conditioned by the MSCs.

7. The method of paragraph 6, wherein the composition does not comprise the media conditioned by the MSCs.

8. The method of any one of paragraphs 1-7, wherein the MSCs are primary cells or a cell line.

9. The method of any one of paragraphs 1-8, wherein the MSCs are from bone barrow, placenta, umbilical cord blood, adipose tissue, adult muscle, conical stroma, or the dental pulp of deciduous teeth.

10. The method of any one of paragraphs 1-9, wherein the MSCs are adipose-derived stem cells.

11. The method of any one of paragraphs 1-10, wherein the extracellular vesicles comprise or consist of ectosomes, microvesicles (MV), microparticles, exosomes, oncosomes, apoptotic bodies (AB), tunneling nanotubes (TNT), or a combination thereof.

12. The method of paragraph 11, wherein the extracellular vesicles comprise or consist of exosomes, microvesicles or a combination thereof.

13. The method of any one of paragraphs 1-12, wherein the extracellular vesicles comprise or consist of a vesicles having a size of between about 20 nm and about 500 nm, or between about 20 nm and about 250 nm, or between about 20 nm and about 200 nm, or between about 20 nm and about 150 nm, or between about 20 nm and about 100 nm.

14. The method of any one of paragraphs 1-13, wherein the extracellular vesicles comprise CD9, CD63, or a combination thereof 15. The method of any one of paragraphs 1-14, wherein the extracellular vesicles comprise one or more of miR-199a-3p, miR-145-5p, miR-143-3p, miR-377-3p, miR-100-3p, miR-29a-3p, miR-495-3p, miR-29c-3p, miR-658, miR-493-3p, miR-184, and miR-27a-3p.

16. The method of any one of paragraphs 1-15 comprising increasing the proliferation, migration, and/or tube formation of lymphatic endothelial cells, increasing expression of one or more lymphatic markers (e.g., hyaluronan receptor-1(LYVE-1), vascular endothelial growth factor receptor-3 (VEGFR-3), prospero homeobox 1 (Prox1), and/or podoplanin) in lymphatic endothelial cells, increasing angiogenesis, increasing lymphangiogeneisis, reducing inflammatory response, decreasing fibrosis formation, enlarging circumference and/or inducing formation of capillary vessels and/or lymphatic vessels, inducing formation of vessels that express both vascular and lymphatic markers, increasing drainage routes (e.g., for accumulated fluids), increasing HIF1-alpha expression and/or activity, reducing Prohibitin (PHB) expression and/or activity, or a combination thereof in the subject.

17. The method of any one of paragraphs 1-16, wherein the subject has a blockage in the lymphatic system, optionally wherein the blockage prevents lymph fluid from draining well, and wherein the fluid buildup leads to swelling.

18. The method of any one of paragraphs 1-17, wherein the subject has one or more symptoms selected from swelling of part or all of the arm(s) and/or leg(s), a feeling of heaviness or tightness, restricted range of motion, aching or discomfort, recurring infections, and fibrosis in one or both arms and/or legs.

19. The method of any one of paragraphs 1-18, wherein the subject has been diagnosed with lymphedema.

20. The method of any one of paragraphs 1-19, wherein the composition is administered by local injection or infusion at or adjacent to a site of interest.

21. The method of paragraph 20, wherein the site of interest is in one or both arms and/or legs.

22. The method of paragraphs 20 or 21, wherein the site of interest is a site of lymphatic blockage and/or lymphedema.

23. The method of any one of paragraphs 1-22, wherein the composition is administered by intramuscular, intraperitoneal, intravenous, subcutaneous, or subdermal injection.

24. The composition of any one of paragraphs 1-23.

25. A composition comprising an effective amount of extracellular vesicles formed by mesenchymal stem cells (MSCs) suitable for use in the method of any one of paragraphs 1-23.

26. Use of the composition of paragraph 24 or 25 for promoting generation or regeneration of the lymphatic system.

27. Use of the composition of paragraph 24 or 25 for the manufacture of a medicament for promoting generation or regeneration of the lymphatic system.

28. Use of the composition of paragraph 24 or 25 for treating lymphedema.

29. Use of the composition of paragraph 24 or 25 for the manufacture of a medicament for treating lymphedema.

30. A composition, use, or method according to any of the disclosure herein including, but not limited to, the description, the experimental examples, and/or the figures and their descriptions.

### EXAMPLES

### Example 1

### Characteristics of ADSC-Derived EVs Materials and Methods

Cell Culture and Preparation of EVs

Human adipose derived stem cells were purchased from Lonza (Basel, Switzerland) and cultured in Dulbecco's Modified Eagle Medium (DMEM; Nissui Pharmaceutical Co, Tokyo, Japan) supplemented with 10% fetal bovine serum. Primary cells were cultured for 7 days (passage 0), replacing the medium three times weekly. Cell passage was done each week in 0.25% trypsin/2 mM EDTA (37° C., 5 min). All ADSCs were used within the sixth passage.

At approximate 80% confluence, ADSCs were washed with PBS thrice and the culture media were replaced with DMEM containing 0.1% fetal bovine serum. After incubation for two days, the medium was collected and centrifuged at 2,000 g for 15 mM at room temperature. To thoroughly remove cellular debris, the supernatant was filtered with a 0.22-mm filter unit (Millipore). Then, the conditioned media (CM) was ultracentrifuged at 110,000 g (35,000 rpm) for 70 mM at 4° C. using an SW41 rotor (Beckman) (Clotilde, et al., J Extracell Vesicles, 7(1):1535750 (2018)). The pellets were washed with 11 ml PBS, and after ultracentrifugation, they were resuspended in PBS. The protein concentration of the putative EV fraction was determined using a Quant-iT Protein Assay with a Qubit 2.0 Fluorometer (Invitrogen). To determine the size distribution of the EVs, nanoparticle tracking analysis was carried out using the Nanosight system (NanoSight) on samples diluted 500- to 1,000-fold with PBS for analysis. The system focuses a laser beam through a suspension of the particles of interest. These are visualized by light scattering using a conventional optical microscope perpendicularly aligned to the beam axis, which collects light scattered from every particle in the field of view. A 60 s video recorded all events for further analysis by the nanoparticle tracking analysis software. The Brownian motion of each particle was tracked between frames to calculate its size using the Stokes-Einstein equation. Using same procedures and culture media, EVs derived from Human Embryonic Kidney 293 (HEK293) cells (KAC co ltd, Japan) were obtained.

PKH67-Labelled EVs Transfer

Purified EVs derived from ADSCs were labelled with a PKH67 green fluorescence labelling kit (Sigma-Aldrich, Mo., USA). EVs were incubated with 2 mM of PKH67 for 5 min and washed five times using a 100 kDa filter (Microcon YM-100, Millipore) to remove excess dye. PKH67-labelled EVs were used to assess EV uptake in vitro.

### Results

Recently, it has been shown that EVs secreted by ADSCs contribute to their paracrine effects. To identify the angiogenic and lymphangiogenic capacity of ADSC-EVs, putative EV fractions were isolated from conditioned media of ADSCs. ADSC-EVs exhibited the characteristic round morphology with bilayer structure under a transmission electron microscope. Nano tracking analysis showed that the size distribution of the isolated EVs was physically homogeneous with a peak diameter of 82 nm (FIG. 1).

Immunoblot analyses showed that tetraspanin CD9 and CD63, reliable exosomal markers, were present in the EV fraction. Fluorescence microscopy analysis demonstrated that the PKH67-labelled EVs had been taken up and was transferred to perinuclear compartments, presumably representative of late endocytic compartments.

### Example 2

### ADSC-Derived EVs have Lymphangiogenic Effects

### Materials and Methods

LEC Culture

Human dermal lymphatic microvascular endothelial cells (HMVEC-dLy Ad) were purchased from Lonza (Basel, Switzerland) and cultured in endothelial growth medium-2-MV(EGM-2-MV; Lonza) that consisted of endothelial basal medium-2 (EBM-2; Lonza) supplemented with 5% fetal bovine serum (FBS), human basic fibroblast growth factor (bFGF), human

VEGF, human insulin like growth factor-1 (IGF-1), human epidermal growth factor, hydrocortisone, ascorbic acid, and gentamicin and amphotericin (SingleQuots; Lonza), according to the manufacturer's instructions. Lymphatic endothelial cells between passages 3 and 6 were used for all experiments in this study.

Proliferation Assay

LEC proliferation assays were performed as previously described (Takeda, et al., Ann Plast Surg., 74(6):728-36 (2015)) LECs were treated with 100 μL of EBM-2 containing PBS, 10 ng/ml recombinant human VEGF-C (rVEGF-C) (R&D Systems, Minneapolis, Minn.), HEK293-EVs, or ADSC-EVs (10 μg/ml) for 48 hours at 37° C. in 5% CO₂. Cell proliferation activity was measured using Cell Counting Kit-8 (Dojindo, Kumamoto, Japan), in which 10 μL of the WST-8 assay solution was added to each well and incubated for 4 hours. The absorbance was measured using a microplate reader (Bio-Rad, Hercules, Calif.) at a wavelength of 450 nm.

Migration Assay

LEC migration assays were performed using Transwell chambers with inserts with 8-μm pores (Corning Costar) as previously described (Takeda, et al., Ann Plast Surg., 74(6):728-36 (2015)). The lower chambers were filled with 700 μL of EBM-2 containing PBS, 10 ng/ml rVEGF-C, HEK293-EVs, or ADSC-EVs (10 μg/ml). After incubation for 16 hours at 37° C. in 5% CO2, the cells on the lower surface of the filter were stained using Diff-Quik (Sysmex, Hyogo, Japan).LECs were photographed through the pores at 100x magnification in 10 random fields, and the migrated cells were counted.

Tube Formation Assay

Matrigel tube formation assays were carried out as previously described (Takeda, et al., Ann Plast Surg., 74(6):728-36 (2015)). LECs were seeded onto the coated wells and cultured in 500 μL of EBM-2 containing PBS, 10 ng/ml rVEGF-C, HEK293-EVs, or ADSC-EVs (10 μg/ml). After incubation for 8 hours at 37° C. in 5% CO₂, tube formation images were captured at 40× magnification in 5 random fields. For quantification, the tube length was measured using NIH ImageJ software.

Statistical Analysis

All statistical analyses were performed using GraphPad Prism 8 (GraphPad Software, Inc., La Jolla, Calif.). All data were expressed as mean±SEM, with the results in the three groups compared by one-way analysis of variance for more than three groups and t test for two groups, with a value of p<0.05 considered significant.

### Results

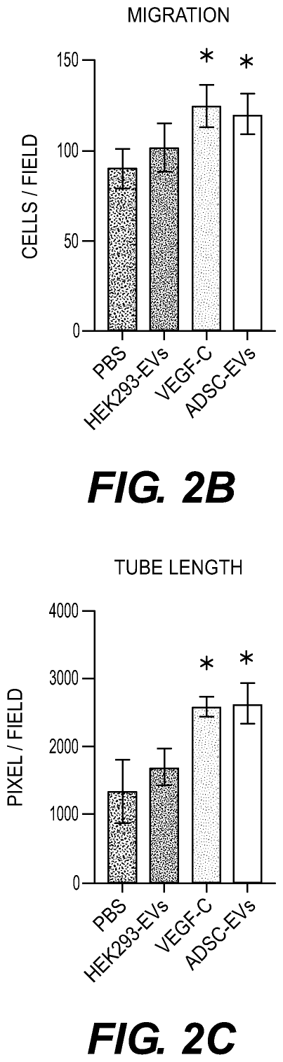

To investigate the ability of ADSC-EVs to enhance lymphangiogenesis in vitro, the proliferation, migration, and tube formation was investigated in LECs, after addition of ADSC-EVs. Proliferation assay showed that ADSC-EVs markedly increased proliferation (absorbance: 1.51+−0.21, P<0.05) by up to one and one-half times compared with PBS treated group (absorbance: 1.095+−0.11). Similar tendency was observed in VEGF-C treated group (absorbance: 1.62+−0.19, P<0.05), although HEK293-EVs treated group showed no significant changes (absorbance: 1.15+−0.20, P>0.05) (FIG. 2A). Migration assay showed that ADSC-EVs significantly promoted LECs migration (120.1+−12.2, cells/field, P<0.05) compared with PBS treated group (90.2 +−11.2, cells/field), as well as VEGF-C treated group (125.3+−11.7, cells/field, P<0.05). HEK293-EVs did not show a significant difference (102.1+−13.3, cells/field, P>0.05) (FIG. 2B). The role of ADSC-EVs was further assessed in the regulation of LECs tube formation. Morphological analysis showed ADSC-EVs promote tube formation (2620.5+−321.3, pixel/field, P<0.05) of LECs compared with PBS treated group (1317.8+−500.8 pixel/field) (FIG. 2C). VEGF-C treated group showed a significant difference (2593.4+−156.2, pixel/field, P<0.05), however, HEK293-EVs showed no significant difference (1702.1+−280.3, pixel/field, P>0.05) (FIG. 2C) These results indicate that ADSC-EVs have lymphangiogenic molecules such as cytokines or miRNAs that stimulated proliferation, migration, and tube formation of LECs.

### Example 3

### qRT-PCR Analysis Shows Increased Expression of Lymphatic Marker mRNA after ADS C-Derived EVs Treatment

### Materials and Methods

qRT-PCR

To assess the effect of ADSC-EVs on LECs, confluent LECs in 24-well plates were treated with 500 μl of EBM-2 containing PBS, rVEGF-C(10 ng/ml), or ADSC-EVs(10 μg/ml) for 12 or 24 hours. Total RNA was extracted from LECs cultured in each condition using a QIAzol and the miRNeasy Mini Kit (Qiagen, Holden, Germany) according to the manufacturer's protocols. For qRT-PCR analysis, complementary DNA was generated from 1 μg of total RNA using a High Capacity cDNA Reverse Transcription Kit (Applied Biosystems). Real-Time PCR system was subsequently performed in triplicate with a 1:15 dilution of cDNA using TaqMan Gene Expression Assays (Applied Biosystems) on a StepOne Real-Time PCR System (Applied Biosystems). Each Assay ID (Thermo Fisher Scientific) is LYVE-1 (Hs00272659_m1), VEFF-R3 (Hs01047677_m1), Prox1 (Hs00896293_m1), and podoplanin (Hs00366766_m1). All mRNA quantification data from cultured cells were normalized to the expression of glyceraldehydes 3-phosphate dehydrogenase (GAPDH).

### Results

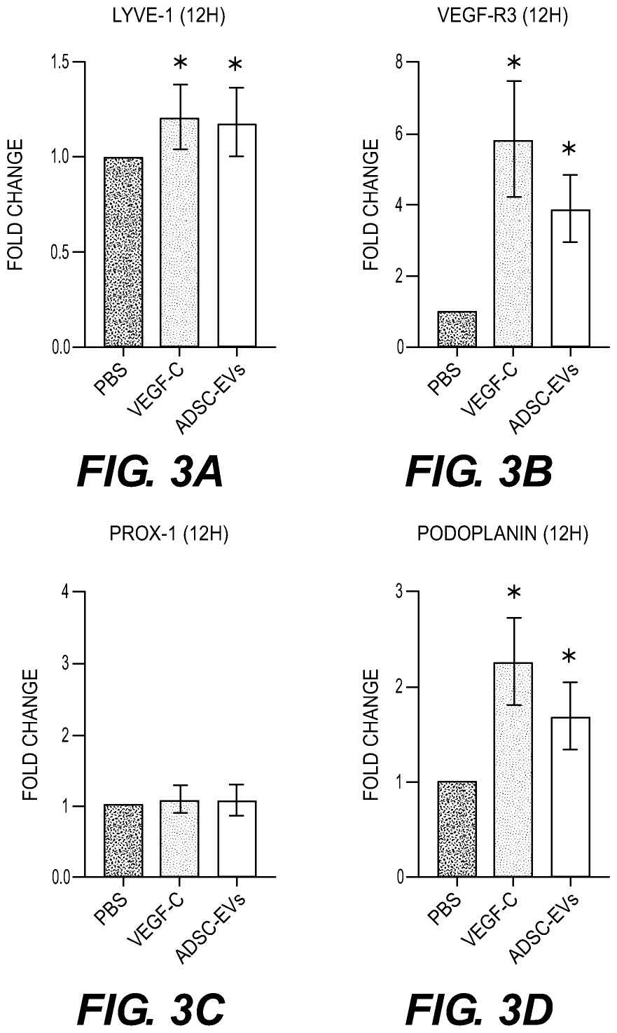

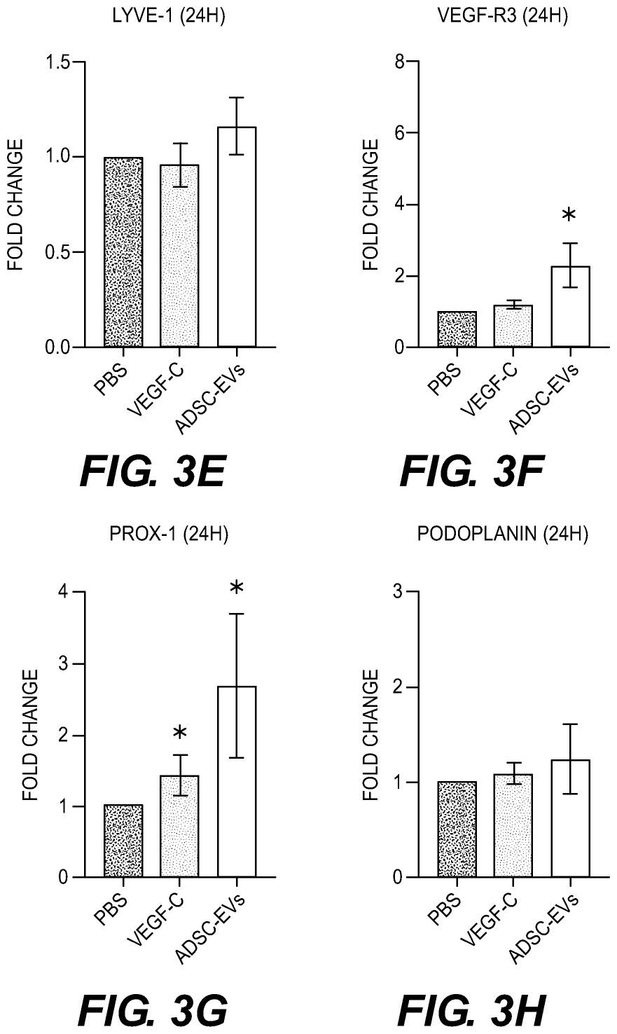

Lymphatic vessel endothelial hyaluronan receptor-1 (LYVE-1), vascular endothelial growth factor receptor-3 (VEGFR-3), prospero homeobox 1 (Prox1), and podoplanin are well known lymphatic markers in LECs. Therefore, expression levels of mRNA of LYVE-1, VEGFR-3, Prox1, and podoplanin in LECs were examined under conditions of PBS, VEGF-C, or ADSC-EVs treatment. The mRNA expression of LYVE-1 was slightly higher in both 12 and 24 hours in the ADSC-EVs treatment group compared to the PBS group. The VEGF-C group showed a similar trend to the ADSC-EVs treatment group. As for VEGFR-3 mRNA expression, it was almost four times higher in 12 hours and twice higher in 24 hours in the ADSC-EVs treatment group than the PBS treatment group. The VEGF-C treated group showed five to six times higher expression than the PBS treated group in 12 hours. The mRNA expression of Prox1 showed no significant differences in 12 hours, however, it was almost 2.5 times higher in the ADSC-EVs treatment group than PBS treatment group, after 24 hours. VEGF-C treatment group showed little expression changes compared to the ADSC-EVs treatment group. The expression levels of Podoplanin were increased 1.5 times higher in ADSC-EVs treatment group than PBS treatment group in 12 hours; however, it showed no significant differences in 24 hours. VEGF-C group showed the same trend as the ADSC-EVs treatment group. See FIGS. 3A-3H.

### Example 4

### ADSC-Derived EV Improved Hindlimbs Appearance in a Lymphedema Mouse Model

### Materials and Methods

Lymphedema Mouse Model, Treatment and Edema Assessment

Lymphedema was established in the bilateral hindlimbs of 10-week-old female C57BL/6J mice (CLEA Japan, Inc., Tokyo, Japan) as previously described (Iwasaki, et al., Plast Reconstr Surg., 139(1):67e-78e (2017)). They were handled according to the guidelines established for animal care at the center and the protocol was approved by the Institutional Animal Care and Use Committee of Jichi Medical University. After gas anesthesia, the mice were subjected to x-ray irradiation in the bilateral inguinal region at 10 Gy in a single dose twice prior to the surgery. The radiation was emitted from an x-ray machine (MX-160Labo, mediXtec, Japan). One week later, mice were subjected to circumferential incision in the inguinal region to the muscle layer. The subiliac and popliteal lymph nodes and the lymphatic vessels entwined around the sciatic veins were removed. To block the superficial lymphatic system, a 3-mm-wide silicone splint was placed in the inguinal wound and then fixed to the skin and underlying muscle using interrupted 6-0 nylon sutures (Bear medic, Tokyo, Japan). This silicon splint placement prevented wound contraction and desiccation. As a therapeutic intervention, injection of PBS (25 μ1) or ADSC-EVs (˜40 μg) or HEK293-EVs (˜40 μg) as a thin layer to the whole leg area was performed in postoperative day 7 and 14. Hindlimb circumference measurements were performed by unblind reviewers at a point 6 mm proximal to the heel at various time points.

Fluorescence lymphography was used to compare the lymphatic structures in swollen hindlimbs treated with PBS or ADSC-EVs every 2 weeks postoperatively using a near-infrared fluorescence camera system (FLUORO, Toa Kogaku, Japan). To examine the thickness of soft tissue postoperatively, we used computed tomography (CT) imaging over 4 weeks postoperatively.

Edema Assessment

To measure the extent of postsurgical edema, hindlimb circumference measurements were performed at a point 6 mm proximal to the heel. In addition, to evaluate the state of lymphedema and the effect of injection of ADSC-EVs, hindlimb circumferences were determined at various time points. Fluorescence lymphography was used to compare lymphatic structures in swollen hindlimbs treated PBS or ADSC-EVs every 2 weeks postoperatively. The animals were clipped and residual hair was removed with a depilatory cream before imaging. Anesthetized mice with isoflurane were placed on a warming pad. A 5-μl volume of a 1-mg/ml solution of indocyanine green (Sigma-Aldrich, St. Louis, Mo.) dissolved in distilled water was injected subcutaneously into the dorsal aspect of both paws using a 26-gauge needle. Fluorescence images were acquired 15 mintes after indocyanine green injection using a near-infrared fluorescence camera system (FLUORO, Toa Kogaku, Japan). Computed tomography (CT) imaging was used to examine the thickness of soft tissue postoperatively, over 4 weeks. Anesthesized mice with isoflurane were placed inside the CT. After the CT images were acquired, the thickness of the soft tissue (length from bone to skin) was measured.

### Results

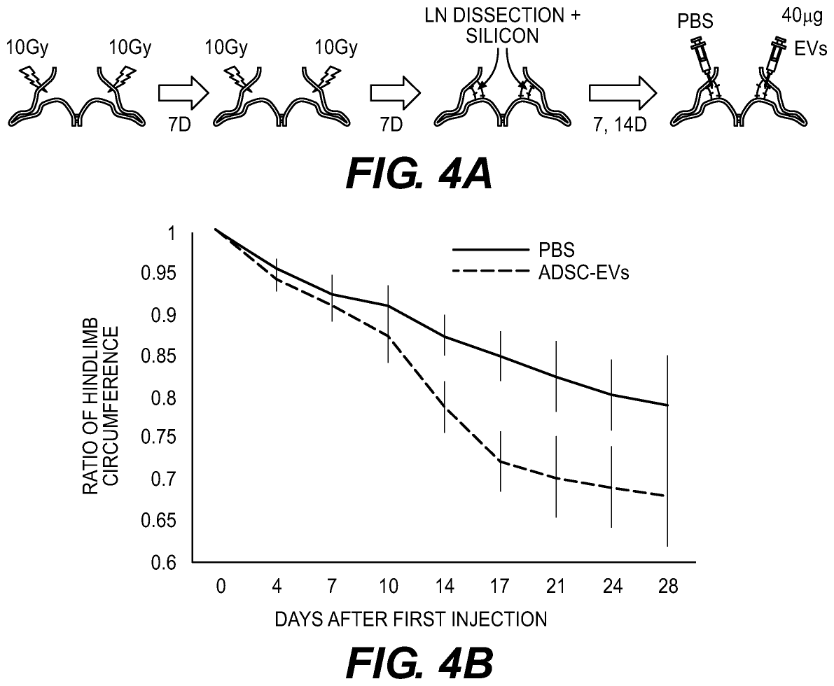

All limbs demonstrated consistent enlargement after radiation, surgical removal of lymph nodes and lymphatic vessels and silicone fixation (FIG. 4A). Although the limb volumes did not return to preoperative levels in these animals, treatment with ADSC-EVs was highly effective in decreasing gross leg swelling (65.1% +−4.5%) as compared with controls (79.4% +−5.1%) (FIG. 4B).

To assess lymphatic function, indocyanine green lymphography of the hindlimb was performed. Using indocyanine green lymphography 4 weeks after the final treatment, clearance of accumulated lymphatic fluid and a clear linear sign of lymphatic channels in ADSC-EVs-treated legs. In contrast, control legs demonstrated pooling of ICG in the whole area and no transport across the zone of surgery (FIG. 4C). This finding was confirmed using computed tomographic (CT) images. The thickness of soft tissue area, which was defined between bone and skin, markedly decreased in the ADSC-EVs treated group compared with the controls, indicating that the excess amount of lymphatic fluid was drained into the venous system.

### Example 5

### ADSC-Derived EVs Promoted Angiogenesis and Lymphangiogenesis in the Affected Limb

### Materials and Methods

Immunohistochemistry

The mice were sacrificed 4 weeks after the final injection of PBS or ADSC-EVs. Skin sections (5 μm) were generated from 6 mm distal to the inguinal wound and 6 mm proximal to the ankle. The tissues were stained with Hoechst 33342 (Dojindo, Tokyo, Japan), lymphatic vessel endothelial receptor-1 (LYVE-1) polyclonal antibodies (bs-1311R-A555, Bioss Antibodies, USA), anti-CD31 polyclonal antibodies (bs-0468R-A647, Bioss Antibodies, USA), SMAD3 polyclonal antibodies (bs-2225R-A488, Bioss Antibodies, USA), and collagen 1 polyclonal antibodies (bs-10423R-A488, Bioss Antibodies, USA). Stained slides were examined under a fluorescence microscope (Keyence, Osaka, Japan). Fluorescence images were captured at 40×magnification in 5 random fields. For quantification, each positive area was measured using NIH ImageJ software by unblind reviewers.

### Results

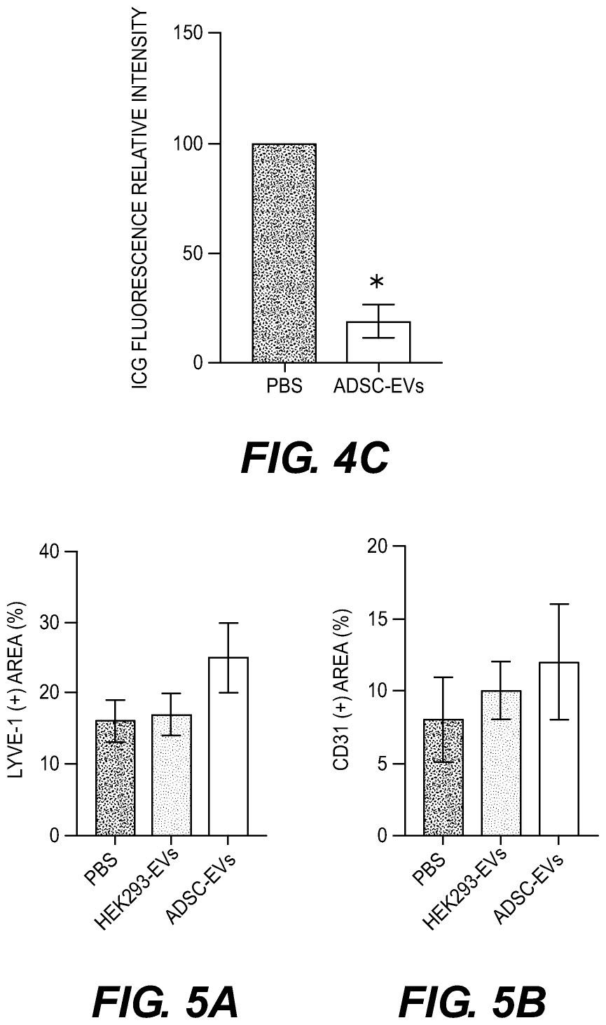

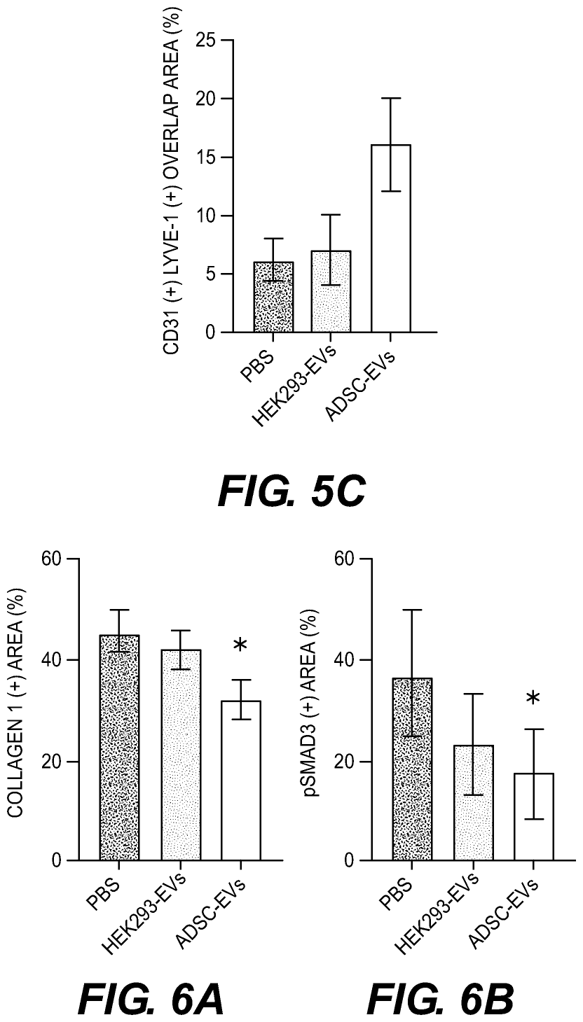

To determine the lymphangiogenic and angiogenic effects of ADSC-EVs in lymphedema leg models in which drainage of lymphatic fluid is obstructed surgically, histologic changes of affected limbs were evaluated. Immunohistochemical analysis of LYVE-1+ vessel counts in the hindlimb tissues of experimental and control legs demonstrated that injection of ADSC-EVs group modestly but significantly increased the total number of capillary lymphatic vessels (25.3%+−5.2%, P<0.05) compared to PBS group and HEK293-EVs group (16.1%+−2.8%, 16.9%+−3.2% respectively) (FIG. 5A). Similarly, analysis of cross-sections obtained from the hindlimb in the mouse models demonstrated increase in the number of CD31+ endothelial cells in the hindlimb tissues treated with ADSC-EVs (12.3%+−3.9%) as compared with PBS or HEK293-EVs (7.7%+−3.3%, 10.2%+−2.2%, respectively) (FIG. 5B). Consistent with the increased number of vascular and lymphatic endothelial cells, an increase of the number of vessels which expressed both CD31+ and LYVE-1+ in tissue samples harvested from the ADSC-EVs(5.2%+−1.1%, P<0.05) group was noted compared to controls (2.1%+−0.2%, 2.3%+−0.3%) (FIG. 5C). These overlapping regions may be the newly connected bypass between lymphatics to capillary vessels, indicating that interstitial transport capacity is greatly increased after ADSC-EVs treatment.

### Example 6

### ADSC-Derived EVs Decreased Fibrosis After Lymphatic Injury

Because the degree of fibrosis in lymphedema patients correlates with the severity of disease, several markers of fibrosis were analyzed in hindlimbs to understand the effects of ADSC-EVs treatment. ADSC-EVs significantly decreased subdermal type I collagen deposition (32.5%+−4.4%, P<0.05) as compared with PBS group (45.8%+−5.1%) and HEK293-EVs group (42.2%+−4.5%) (FIG. 6A). ADSC-EVs treatment significantly decreased expression of phosphorylated SMAD3 (pSMAD-3) (17.1%+−9.2%, P<0.05), which is a downstream signaling molecule of pro-fibrotic growth factor, compared to PBS group(36.5%+−14.2%) and HEK293-EVs group (23.3%+−10.4%) (FIG. 6B). Taken together, these findings show that ADSC-EVs mitigate the formation of fibrotic response in the setting of lymphedema.

### Example 7

### ADSC-Derived EVs Contain Several miRNAs Targeting MDM2, HIF1a, and PHB

### Materials and Methods

miRNA Expression Profiling by Real-Time PCR Arrays

Total RNA, including miRNA, was extracted from ADSC-EVs and HEK293-EVs using Trizol reagent (Invitrogen Life Technologies, Carlsbad, CA, USA) according to the manufacturer's instruction. From each sample, 15 μl of miRNAs were reverse-transcribed using a miRCURY LNA RT Kit (Qiagen, Netherlands). The cDNA was mixed with 2× PCR master mix (Qiagen, Netherlands). All the real-time PCR-based experiments using a

LightCycler 480 Instrument 2 according to the manufacturer's instructions. For normalization, miRNA expressions were compared between ADSC-EVs and HEK293-EVs. The cycle threshold (2-AACt) method was utilized to calculate the fold change.

mRNA Expression Profiling in LECs by Microarray

To perform a mRNA microarray, 5×10e4 LECs were seeded into 24-well plates and 12 h later EVs from ADSC or PBS as control were added to LECs. Total mRNAs were extracted from LECs 12 h after treatment with the EVs and PBS. For each hybridization, 0.60 μg of Cy3 labeled cRNA were fragmented, and hybridized at 65 degrees for 17 hours to an Agilent SurePrint G3 Human GE v3 8×60K Microarray (Design ID: 072363). After washing, microarrays were scanned using an Agilent DNA microarray scanner.

Intensity values of each scanned feature were quantified using Agilent feature extraction software version 11.5.1.1, which performs background subtractions. Only features that were flagged as no errors (Detected flags) were used and features that were not positive, not significant, not uniform, not above background, saturated, and population outliers (Not Detected and Compromised flags) were excluded. Normalization was performed using Agilent GeneSpring software version 14.9.1 (per chip:normalization to Quantile).

### Results

To determine how ADSC-EVs regulate miRNAs associated with various processes, using a real-time RT-PCR-based miRNA array, the expression patterns of 752 different miRNAs were next profiled in ADSC-EVs. The expression patterns of the same miRNAs in HEK293 cells derived EVs were also analyzed as controls. The miRNA PCR array revealed that 396 differentially expressed miRNAs in ADSC-EVs compared to HEK293-EVs. miRNAs expressed over double fold in log2 ratio are shown in Table 1.

Bioinformatic analysis (Ingenuity Pathway Analysis, IPA), was performed to evaluate the implications of the altered miRNA expression. IPA revealed that the major networks which incorporate the predicted targets, include functions such as ‘Organismal injury and abnormalities’, ‘Reproductive system disease’, ‘Inflammatory disease’, and ‘Inflammatory response’.

The cargo of EVs, which includes miRNAs, mRNAs, proteins and other biological components, is transferred from the donor to recipient cells and influences the cellular phenotypes. Therefore, which genes are altered in the LECs treated with the ADSC-EVs or PBS as control were investigated.

Total RNA was extracted from treated LECs, and microarray analyses were performed. Results show that differentially expressed genes among treatments with ADSC-EVs and control PBS, there were clear differences in gene expression in the LECs.

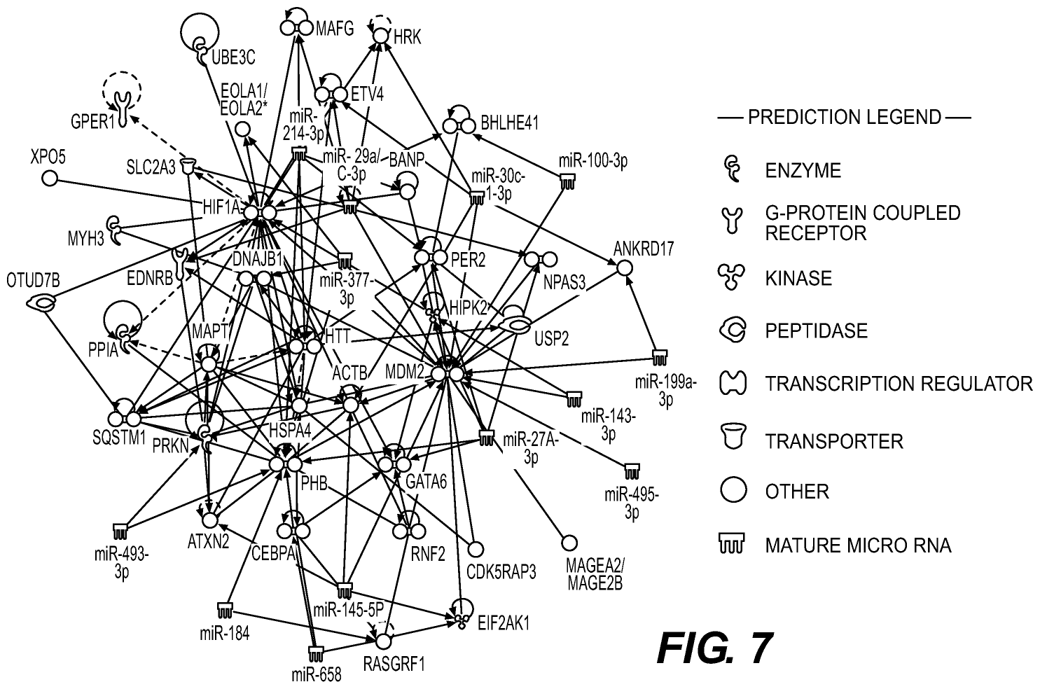

A bioinformatic analysis was conducted using IPA for the relation between highly expressed miRNAs in ADSC-EVs and genes which showed expression changes in LECs after addition of ADSC-EVs (FIG. 7, Table 2). As a result, eight miRNAs such as miR-199a-3p, miR-145-5p, miR-143-3p, miR-377-3p, miR-100-3p, miR-29a-3p, miR-495-3p, and miR-29c-3p were found to target and regulate the expression of mouse double minute 2 homolog (MDM2), which contributed to the stability of hypoxiainducible factor-1 alpha (HIF1a) and resulted in angiogenesis and lymphangiogenesis in LECs.. MDM2 is a cellular oncoprotein encoded by a gene located on chromosome 12q13-14. MDM2 can suppress p53, a cancer suppressor gene. Inhibition of MDM2 has anti-inflammatory effects and may lead to treatment of autoimmune diseases and cancer. Previous studies have shown that MDM2 negatively regulates the stability of hypoxia-inducible factor-1 alpha (HIF1a) in protein level. This investigation found that the expression level of HIF1a was elevated in LECs after addition of ADSC-EVs. HIF1a is a transcription factor that controls the cellular response to hypoxia. HIF1a promotes transcription of various proteins such as VEGF, Erythropoietin (EPO) and glucose transporters, and also plays a key role in lymphangiogenesis and angiogenesis. The result indicates that the reduced expression of MDM2 caused by miR-199a-3p, miR-145-5p, miR-143-3p, miR-377-3p, miR-100-3p, miR-29a-3p, miR-495-3p, and miR-29c-3p in ADSC-EVs lead to elevation of HIF1a in LECs, which leads to angiogenesis and lymphangiogenesis.

Another target of four miRNAs such as miR-658, miR-493-3p, miR-184, and miR-27a-3p in ADSC-EVs was found to be Prohibitin (PHB). PHB is a membrane protein which regulates a variety of biological processes such as apoptosis, cell cycle, signal transduction and cellular senescence. The downregulation of PHB can lead to angiogenesis (Qian, et al., CNS Neurosci Ther., 19(10):804-12 (2013)).

TABLE 1
miRNAs expressed over double fold in log2 ratio

| SEQ ID # | miRname | Log2 Ratio | target sequence |
|---|---|---|---|
| 1 | hsa-miR-548c-3p | 13.1573 | CAAAAAUCUCAAUUACUUUUGC |
| 2 | hsa-miR-214-5p | 12.4406 | UGCCUGUCUACACUUGCUGUGC |
| 3 | hsa-miR-372-3p | 12.4273 | AAAGUGCUGCGACAUUUGAGCGU |
| 4 | hsa-miR-206 | 12.0473 | UGGAAUGUAAGGAAGUGUGUGG |
| 5 | hsa-miR-337-5p | 11.2373 | GAACGGCUUCAUACAGGAGUU |
| 6 | hsa-miR-199a-5p | 10.2440 | CCCAGUGUUCAGACUACCUGUUC |
| 7 | hsa-miR-214-3p | 9.2040 | ACAGCAGGCACAGACAGGCAGU |
| 8 | hsa-miR-199a-3p | 8.9240 | ACAGUAGUCUGCACAUUGGUUA |
| 9 | hsa-miR-370-3p | 8.4440 | GCCUGCUGGGGUGGAACCUGGU |
| 10 | hsa-miR-100-5p | 7.2940 | AACCCGUAGAUCCGAACUUGUG |
| 11 | hsa-miR-145-5p | 7.2440 | GUCCAGUUUUCCCAGGAAUCCCU |
| 12 | hsa-miR-199b-5p | 7.1940 | CCCAGUGUUUAGACUAUCUGUUC |
| 13 | hsa-miR-143-3p | 7.1640 | UGAGAUGAAGCACUGUAGCUC |
| 14 | hsa-miR-888-5p | 7.0240 | UACUCAAAAAGCUGUCAGUCA |
| 15 | hsa-let-71-3p | 6.8206 | CUGCGCAAGCUACUGCCUUGCU |
| 16 | hsa-let-7 a-2-3p | 6.7106 | CUGUACAGCCUCCUAGCUUUCC |
| 17 | hsa-miR-137 | 6.6740 | UUAUUGCUUAAGAAUACGCGUAG |
| 18 | hsa-miR-337-3p | 6.4640 | CUCCUAUAUGAUGCCUUUCUUC |
| 19 | hsa-miR-145-3p | 6.3173 | GGAUUCCUGGAAAUACUGUUCU |
| 20 | hsa-miR-133b | 6.2640 | UUUGGUCCCCUUCAACCAGCUA |
| 21 | hsa-miR-208b-3p | 5.7706 | AUAAGACGAACAAAAGGUUUGU |
| 22 | hsa-miR-493-5p | 5.6873 | UUGUACAUGGUAGGCUUUCAUU |
| 23 | hsa-let-7b-3p | 5.6073 | CUAUACAACCUACUGCCUUCCC |
| 24 | hsa-let-71-5p | 5.6040 | UGAGGUAGUAGUUUGUGCUGUU |
| 25 | hsa-miR-143-5p | 5.5773 | GGUGCAGUGCUGCAUCUCUGGU |
| 26 | hsa-miR-675-5p | 5.4673 | UGGUGCGGAGAGGGCCCACAGUG |
| 27 | hsa-miR-136-3p | 5.4473 | CAUCAUCGUCUCAAAUGAGUCU |
| 28 | hsa-miR-133a-3p | 5.3740 | UUUGGUCCCCUUCAACCAGCUG |
| 29 | hsa-miR-377-5p | 5.2406 | AGAGGUUGCCCUUGGUGAAUUC |
| 30 | hsa-let-7b-5p | 5.2340 | UGAGGUAGUAGGUUGUGUGGUU |
| 31 | hsa-miR-377-3p | 5.1840 | AUCACACAAAGGCAACUUUUGU |
| 32 | hsa-miR-136-5p | 5.1840 | ACUCCAUUUGUUUUGAUGAUGGA |
| 33 | hsa-miR-125b-1-3p | 5.0673 | ACGGGUUAGGCUCUUGGGAGCU |
| 34 | hsa-miR-100-3p | 5.040584 | CAAGCUUGUAUCUAUAGGUAUG |
| 35 | hsa-miR-125b-5p | 4.9640 | UCCCUGAGACCCUAACUUGUGA |
| 36 | hsa-miR-127-3p | 4.8840 | UCGGAUCCGUCUGAGCUUGGCU |
| 37 | hsa-miR-29a-5p | 4.8673 | ACUGAUUUCUUUUGGUGUUCAG |
| 38 | hsa-miR-135a-3p | 4.8106 | UAUAGGGAUUGGAGCCGUGGCG |
| 39 | hsa-miR-539-5p | 4.7340 | GGAGAAAUUAUCCUUGGUGUGU |
| 40 | hsa-miR-424-3p | 4.5773 | CAAAACGUGAGGCGCUGCUAU |
| 41 | hsa-miR-369-5p | 4.5540 | AGAUCGACCGUGUUAUAUUCGC |
| 42 | hsa-miR-376a-3p | 4.2940 | AUCAUAGAGGAAAAUCCACGU |
| 43 | hsa-miR-410-3p | 4.2340 | AAUAUAACACAGAUGGCCUGU |
| 44 | hsa-miR-376c-3p | 4.1940 | AACAUAGAGGAAAUUCCACGU |
| 45 | hsa-miR-154-5p | 4.1840 | UAGGUUAUCCGUGUUGCCUUCG |
| 46 | hsa-miR-409-3p | 4.1740 | GAAUGUUGCUCGGUGAACCCCU |
| 47 | hsa-miR-146a-5p | 4.0940 | UGAGAACUGAAUUCCAUGGGUU |
| 48 | hsa-miR-29a-3p | 4.0640 | UAGCACCAUCUGAAAUCGGUUA |
| 49 | hsa-miR-127-5p | 3.9640 | CUGAAGCUCAGAGGGCUCUGAU |
| 50 | hsa-miR-382-5p | 3.9540 | GAAGUUGUUCGUGGUGGAUUCG |
| 51 | hsa-miR-409-5p | 3.9273 | AGGUUACCCGAGCAACUUUGCAU |
| 52 | hsa-miR-424-5p | 3.8540 | CAGCAGCAAUUCAUGUUUUGAA |
| 53 | hsa-let-7f-1-3p | 3.7873 | CUAUACAAUCUAUUGCCUUCCC |
| 54 | hsa-miR-485-3p | 3.7140 | GUCAUACACGGCUCUCCUCUCU |
| 55 | hsa-miR-369-3p | 3.6273 | AAUAAUACAUGGUUGAUCUUU |
| 56 | hsa-miR-376a-5p | 3.6173 | GUAGAUUCUCCUUCUAUGAGUA |
| 57 | hsa-miR-1 | 3.5940 | UGGAAUGUAAAGAAGUAUGUAU |
| 58 | hsa-let-7d-3p | 3.5340 | CUAUACGACCUGCUGCCUUUCU |
| 59 | hsa-miR-382-3p | 3.4973 | AAUCAUUCACGGACAACACUU |
| 60 | hsa-miR-668-3p | 3.4840 | UGUCACUCGGCUCGGCCCACUAC |
| 61 | hsa-miR-487b-3p | 3.4340 | AAUCGUACAGGGUCAUCCACUU |
| 62 | hsa-miR-411-5p | 3.4240 | UAGUAGACCGUAUAGCGUACG |
| 63 | hsa-miR-544a | 3.4073 | AUUCUGCAUUUUUAGCAAGUUC |
| 64 | hsa-miR-889-3p | 3.4073 | UUAAUAUCGGACAACCAUUGU |
| 65 | hsa-miR-329-3p | 3.2840 | AACACACCUGGUUAACCUCUUU |
| 66 | hsa-miR-503-5p | 3.2540 | UAGCAGCGGGAACAGUUCUGCAG |
| 67 | hsa-miR-30c-1-3p | 3.2473 | CUGGGAGAGGGUUGUUUACUCC |
| 68 | hsa-miR-22-5p | 3.1840 | AGUUCUUCAGUGGCAAGCUUUA |
| 69 | hsa-miR-658 | 3.1473 | GGCGGAGGGAAGUAGGUCCGUUGGU |
| 70 | hsa-miR-1185-5p | 3.1773 | AGAGGAUACCCUUUGUAUGUU |
| 71 | hsa-miR-495-3p | 3.1240 | AAACAAACAUGGUGCACUUCUU |
| 72 | hsa-miR-432-5p | 3.1040 | UCUUGGAGUAGGUCAUUGGGUGG |
| 73 | hsa-miR-379-5p | 3.1040 | UGGUAGACUAUGGAACGUAGG |
| 74 | hsa-miR-22-3p | 3.0640 | AAGCUGCCAGUUGAAGAACUGU |
| 75 | hsa-miR-654-3p | 3.0373 | UAUGUCUGCUGACCAUCACCUU |
| 76 | hsa-miR-365b-5p | 3.0273 | AGGGACUUUCAGGGGCAGCUGU |
| 77 | hsa-miR-493-3p | 2.9740 | UGAAGGUCUACUGUGUGCCAGG |
| 78 | hsa-miR-134-5p | 2.9340 | UGUGACUGGUUGACCAGAGGGG |
| 79 | hsa-miR-154-3p | 2.8740 | AAUCAUACACGGUUGACCUAUU |
| 80 | hsa-miR-411-3p | 2.8273 | UAUGUAACACGGUCCACUAACC |
| 81 | hsa-miR-335-3p | 2.7773 | UUUUUCAUUAUUGCUCCUGACC |
| 82 | hsa-miR-543 | 2.7573 | AAACAUUCGCGGUGCACUUCUU |
| 83 | hsa-miR-381-3p | 2.6940 | UAUACAAGGGCAAGCUCUCUGU |
| 84 | hsa-miR-574-3p | 2.6640 | CACGCUCAUGCACACACCCACA |
| 85 | hsa-miR-379-3p | 2.6173 | UAUGUAACAUGGUCCACUAACU |
| 86 | hsa-miR-21-5p | 2.6040 | UAGCUUAUCAGACUGAUGUUGA |
| 87 | hsa-miR-654-5p | 2.6040 | UGGUGGGCCGCAGAACAUGUGC |
| 88 | hsa-miR-605-5p | 2.5573 | UAAAUCCCAUGGUGCCUUCUCCU |
| 89 | hsa-miR-29c-3p | 2.5440 | UAGCACCAUUUGAAAUCGGUUA |
| 90 | hsa-miR-184 | 2.5040 | UGGACGGAGAACUGAUAAGGGU |
| 91 | hsa-miR-193a-5p | 2.4940 | UGGGUCUUUGCGGGCGAGAUGA |
| 92 | hsa-miR-541-5p | 2.4773 | AAAGGAUUCUGCUGUCGGUCCCACU |
| 93 | hs a-miR-27 a-3p | 2.4740 | UUCACAGUGGCUAAGUUCCGC |
| 94 | hsa-miR-494-3p | 2.4240 | UGAAACAUACACGGGAAACCUC |
| 95 | hsa-miR-376b-3p | 2.4140 | AUCAUAGAGGAAAAUCCAUGUU |
| 96 | hsa-miR- 144-5p | 2.3673 | GGAUAUCAUCAUAUACUGUAAG |
| 97 | hsa-miR-1244 | 2.3473 | AAGUAGUUGGUUUGUAUGAGAUGGUU |
| 98 | hsa-miR-323a-3p | 2.3440 | CACAUUACACGGUCGACCUCU |
| 99 | hsa-miR-335-5p | 2.2940 | UCAAGAGCAAUAACGAAAAAUGU |
| 100 | hsa-miR-21-3p | 2.2640 | CAACACCAGUCGAUGGGCUGU |
| 101 | hs a-let-7 d-5p | 2.0940 | AGAGGUAGUAGGUUGCAUAGUU |
| 102 | hsa-miR-487 a-3p | 2.0773 | AAUCAUACAGGGACAUCCAGUU |

TABLE 2
Differentially expressed genes in LEC for treatment of EVs or PBS

| | | | Fold | Log fold |
|---|---|---|---|---|
| | | Expression | change | change |
| | | compared to | (ADSC EV | (ADSC EV |
| GeneSymbol | GeneName | control | vs PBS) | vs PBS) |
| FOXB1 | forkhead box B1 | up | 19.85 | 4.31 |
| WWC2-AS1 | WWC2 antisense RNA 1 | up | 19.66 | 4.30 |
| CFAP61 | cilia and flagella associated | up | 14.71 | 3.88 |
| | protein 61 | | | |
| CHST10 | carbohydrate sulfotransferase 10 | up | 13.57 | 3.76 |
| HRK | harakiri, BCL2 interacting | up | 12.81 | 3.68 |
| | protein | | | |
| KLK8 | kallikrein-related peptidase 8 | up | 12.43 | 3.64 |
| MYHAS | myosin heavy chain gene cluster | up | 12.15 | 3.60 |
| | antisense RNA | | | |
| HTR4 | 5-hydroxytryptamine (serotonin) | up | 11.96 | 3.58 |
| | receptor 4, G protein-coupled | | | |
| ANAPC1 | anaphase promoting complex | up | 10.64 | 3.41 |
| | subunit 1 | | | |
| LYPD6B | LY6/PLAUR domain containing | up | 9.75 | 3.29 |
| | 6B | | | |
| LIG3 | ligase III, DNA, ATP-dependent | up | 9.75 | 3.29 |
| TSPEAR | thrombospondin-type laminin G | up | 9.41 | 3.23 |
| | domain and EAR repeats | | | |
| OR6A2 | olfactory receptor, family 6, | up | 9.28 | 3.21 |
| | subfamily A, member 2 | | | |
| GRIN2C | glutamate receptor, ionotropic, | up | 9.21 | 3.20 |
| | N-methyl D-aspartate 2C | | | |
| GPR141 | G protein-coupled receptor 141 | up | 9.11 | 3.19 |
| MUC7 | mucin 7, secreted | up | 8.51 | 3.09 |
| ATXN8OS | ATXN8 opposite strand (non- | up | 8.14 | 3.03 |
| | protein coding) | | | |
| KLK13 | kallikrein-related peptidase 13 | up | 8.05 | 3.01 |
| ADAMTS8 | ADAM metallopeptidase with | up | 8.01 | 3.00 |
| | thrombospondin type 1 motif, 8 | | | |
| ILF2 | interleukin enhancer binding | up | 7.59 | 2.92 |
| | factor 2 | | | |
| RMDN2-AS1 | RMDN2 antisense RNA 1 | up | 7.48 | 2.90 |
| CD300C | CD300c molecule | up | 7.44 | 2.90 |
| LRP1 | low density lipoprotein receptor- | up | 7.25 | 2.86 |
| | related protein 1 | | | |
| CCDC13 | coiled-coil domain containing 13 | up | 6.77 | 2.76 |
| POLM | polymerase (DNA directed), mu | up | 6.40 | 2.68 |
| GPER1 | G protein-coupled estrogen | up | 6.37 | 2.67 |
| | receptor 1 | | | |
| SNORD77 | small nucleolar RNA, C/D box | up | 6.14 | 2.62 |
| | 77 | | | |
| SDC2 | syndecan 2 | up | 5.78 | 2.53 |
| KRTAP22-1 | keratin associated protein 22-1 | up | 5.76 | 2.53 |
| SLITRK2 | SLIT and NTRK-like family, | up | 5.72 | 2.52 |
| | member 2 | | | |
| LRRC7 | leucine rich repeat containing 7 | up | 5.51 | 2.46 |
| CHRNA2 | cholinergic receptor, nicotinic, | up | 5.40 | 2.43 |
| | alpha 2 (neuronal) | | | |
| SEPT3 | septin 3 | up | 5.22 | 2.39 |
| TSRM | zinc finger domain-related | up | 5.14 | 2.36 |
| | protein TSRM | | | |
| TRAPPC2 | trafficking protein particle | up | 5.03 | 2.33 |
| | complex 2 | | | |
| WDR5B | WD repeat domain 5B | up | 4.97 | 2.31 |
| PPP1R26-AS1 | PPP1R26 antisense RNA 1 | up | 4.79 | 2.26 |
| SAMD13 | sterile alpha motif domain | up | 4.71 | 2.23 |
| | containing 13 | | | |
| RNF180 | ring finger protein 180 | up | 4.69 | 2.23 |
| ZNF888 | zinc finger protein 888 | up | 4.39 | 2.14 |
| PTCD2 | pentatricopeptide repeat domain | up | 4.38 | 2.13 |
| | 2 | | | |
| TM4SF5 | transmembrane 4 L six family | up | 4.37 | 2.13 |
| | member 5 | | | |
| PHGR1 | proline/histidine/glycine-rich 1 | up | 4.33 | 2.11 |
| LRRC10 | leucine rich repeat containing 10 | up | 4.27 | 2.09 |
| COL27A1 | collagen, type XXVII, alpha 1 | up | 4.26 | 2.09 |
| XKR7 | XK, Kell blood group complex | up | 4.16 | 2.06 |
| | subunit-related family, member 7 | | | |
| NOS1AP | nitric oxide synthase 1 (neuronal) | up | 4.11 | 2.04 |
| | adaptor protein | | | |
| ITLN2 | intelectin 2 | up | 4.11 | 2.04 |
| PTCD2 | pentatricopeptide repeat domain | up | 4.05 | 2.02 |
| | 2 | | | |
| KRT8 | keratin 8, type II | up | 3.92 | 1.97 |
| FAM150B | family with sequence similarity | up | 3.85 | 1.94 |
| | 150, member B | | | |
| SNORD19B | small nucleolar RNA, C/D box | up | 3.83 | 1.94 |
| | 19B | | | |
| MEGF10 | multiple EGF-like-domains 10 | up | 3.76 | 1.91 |
| LRIG2 | leucine-rich repeats and | up | 3.67 | 1.87 |
| | immunoglobulin-like domains 2 | | | |
| LRIT1 | leucine-rich repeat, | up | 3.62 | 1.86 |
| | immunoglobulin-like and | | | |
| | transmembrane domains 1 | | | |
| OR1L8 | olfactory receptor, family 1, | up | 3.56 | 1.83 |
| | subfamily L, member 8 | | | |
| CBX3P2 | chromobox homolog 3 | up | 3.54 | 1.83 |
| | pseudogene 2 | | | |
| RAB11FIP4 | RAB11 family interacting | up | 3.50 | 1.81 |
| | protein 4 (class II) | | | |
| FSD1L | fibronectin type III and SPRY | up | 3.48 | 1.80 |
| | domain containing 1-like | | | |
| ROR1 | receptor tyrosine kinase-like | up | 3.45 | 1.79 |
| | orphan receptor 1 | | | |
| CCDC17 | coiled-coil domain containing 17 | up | 3.42 | 1.78 |
| SLC7A14 | solute carrier family 7, member | up | 3.42 | 1.77 |
| | 14 | | | |
| KCNS1 | potassium voltage-gated channel, | up | 3.41 | 1.77 |
| | modifier subfamily S, member 1 | | | |
| PRY2 | PTPN13-like, Y-linked 2 | up | 3.40 | 1.76 |
| TLCD2 | TLC domain containing 2 | up | 3.40 | 1.76 |
| CEP85L | centrosomal protein 85 kDa-like | up | 3.34 | 1.74 |
| CDC42EP3 | CDC42 effector protein (Rho | up | 3.29 | 1.72 |
| | GTPase binding) 3 | | | |
| PVRL3-AS1 | PVRL3 antisense RNA 1 | up | 3.24 | 1.69 |
| NT5DC4 | 5′-nucleotidase domain | up | 3.23 | 1.69 |
| | containing 4 | | | |
| CNOT4 | CCR4-NOT transcription | up | 3.18 | 1.67 |
| | complex, subunit 4 | | | |
| POFUT1 | protein O-fucosyltransferase 1 | up | 3.17 | 1.67 |
| PTGER4P2- | PTGER4P2-CDK2AP2P2 | up | 3.17 | 1.66 |
| CDK2AP2P2 | readthrough transcribed | | | |
| | pseudogene | | | |
| NCOA2 | nuclear receptor coactivator 2 | up | 3.13 | 1.65 |
| L2HGDH | L-2-hydroxyglutarate | up | 3.12 | 1.64 |
| | dehydrogenase | | | |
| SIRPG | signal-regulatory protein gamma | up | 3.10 | 1.63 |
| MID1 | midline 1 | up | 3.10 | 1.63 |
| WDR55 | WD repeat domain 55 | up | 3.10 | 1.63 |
| GATA6 | GATA binding protein 6 | up | 3.09 | 1.63 |
| RNF216 | ring finger protein 216 | up | 3.06 | 1.61 |
| CGN | cingulin | up | 3.04 | 1.60 |
| SERPINI1 | serpin peptidase inhibitor, clade I | up | 3.01 | 1.59 |
| | (neuroserpin), member 1 | | | |
| LCP1 | lymphocyte cytosolic protein 1 | up | 3.01 | 1.59 |
| | (L-plastin) | | | |
| NR2F2-AS1 | NR2F2 antisense RNA 1 | up | 3.00 | 1.59 |
| FLJ43315 | asparagine synthetase | up | 2.99 | 1.58 |
| | pseudogene | | | |
| AADAC | arylacetamide deacetylase | up | 2.99 | 1.58 |
| NDUFA6-AS1 | NDUFA6 antisense RNA 1 (head | up | 2.98 | 1.57 |
| | to head) | | | |
| PMFBP1 | polyamine modulated factor 1 | up | 2.96 | 1.57 |
| | binding protein 1 | | | |
| RBM23 | RNA binding motif protein 23 | up | 2.96 | 1.57 |
| CALML3-AS1 | CALML3 antisense RNA 1 | up | 2.96 | 1.56 |
| CSRNP3 | cysteine-serine-rich nuclear | up | 2.96 | 1.56 |
| | protein 3 | | | |
| PCDHGC3 | protocadherin gamma subfamily | up | 2.95 | 1.56 |
| | C, 3 | | | |
| CCDC110 | coiled-coil domain containing | up | 2.95 | 1.56 |
| | 110 | | | |
| SYDE2 | synapse defective 1, Rho | up | 2.94 | 1.56 |
| | GTPase, homolog 2 (C. elegans) | | | |
| LRRC7 | leucine rich repeat containing 7 | up | 2.92 | 1.54 |
| SYNGAP1 | synaptic Ras GTPase activating | up | 2.91 | 1.54 |
| | protein 1 | | | |
| MMP25 | matrix metallopeptidase 25 | up | 2.88 | 1.52 |
| PLXDC1 | plexin domain containing 1 | up | 2.87 | 1.52 |
| SPIN3 | spindlin family, member 3 | up | 2.87 | 1.52 |
| SP2-AS1 | SP2 antisense RNA 1 | up | 2.87 | 1.52 |
| FAM178B | family with sequence similarity | up | 2.87 | 1.52 |
| | 178, member B | | | |
| FAM104B | family with sequence similarity | up | 2.86 | 1.52 |
| | 104, member B | | | |
| SSU72 | SSU72 RNA polymerase II CTD | up | 2.86 | 1.52 |
| | phosphatase homolog | | | |
| | (S. cerevisiae) | | | |
| CALY | calcyon neuron-specific vesicular | up | 2.86 | 1.52 |
| | protein | | | |
| ACVR1B | activin A receptor, type IB | up | 2.84 | 1.51 |
| KCTD21-AS1 | KCTD21 antisense RNA 1 | up | 2.84 | 1.50 |
| GPR173 | G protein-coupled receptor 173 | up | 2.84 | 1.50 |
| LBX1-AS1 | LBX1 antisense RNA 1 (head to | up | 2.83 | 1.50 |
| | head) | | | |
| SPDYE3 | speedy/RINGO cell cycle | up | 2.82 | 1.50 |
| | regulator family member E3 | | | |
| CHRDL1 | chordin-like 1 | up | 2.82 | 1.50 |
| CDK20 | cyclin-dependent kinase 20 | up | 2.80 | 1.49 |
| SLC51B | solute carrier family 51, beta | up | 2.80 | 1.48 |
| | subunit | | | |
| ZBED3-AS1 | ZBED3 antisense RNA 1 | up | 2.78 | 1.48 |
| ETV4 | ets variant 4 | up | 2.77 | 1.47 |
| KLHL15 | kelch-like family member 15 | up | 2.75 | 1.46 |
| TCTE3 | t-complex-associated-testis- | up | 2.75 | 1.46 |
| | expressed 3 | | | |
| MAGEA2B | melanoma antigen family A, 2B | up | 2.74 | 1.46 |
| SP2-AS1 | SP2 antisense RNA 1 | up | 2.74 | 1.46 |
| RPAP2 | RNA polymerase II associated | up | 2.74 | 1.45 |
| | protein 2 | | | |
| ITPK1-AS1 | ITPK1 antisense RNA 1 | up | 2.73 | 1.45 |
| ATP1A1-AS1 | ATP1A1 antisense RNA 1 | up | 2.73 | 1.45 |
| C2orf15 | chromosome 2 open reading | up | 2.72 | 1.44 |
| | frame 15 | | | |
| UBE2E4P | ubiquitin-conjugating enzyme | up | 2.71 | 1.44 |
| | E2E 4 pseudogene | | | |
| OCLN | occludin | up | 2.71 | 1.44 |
| CHAC1 | ChaC glutathione-specific | up | 2.70 | 1.43 |
| | gamma-glutamylcyclotransferase | | | |
| | 1 | | | |
| SPOPL | speckle-type POZ protein-like | up | 2.69 | 1.43 |
| TMEM200B | transmembrane protein 200B | up | 2.68 | 1.42 |
| TMEM184B | transmembrane protein 184B | up | 2.68 | 1.42 |
| GLTPD2 | glycolipid transfer protein | up | 2.65 | 1.41 |
| | domain containing 2 | | | |
| OBP2B | odorant binding protein 2B | up | 2.64 | 1.40 |
| CYP4Z1 | cytochrome P450, family 4, | up | 2.64 | 1.40 |
| | subfamily Z, polypeptide 1 | | | |
| CASC11 | cancer susceptibility candidate | up | 2.63 | 1.40 |
| | 11 (non-protein coding) | | | |
| AHCTF1 | AT hook containing transcription | up | 2.63 | 1.40 |
| | factor 1 | | | |
| PFKFB2 | 6-phosphofructo-2- | up | 2.62 | 1.39 |
| | kinase/fructose-2,6- | | | |
| | biphosphatase 2 | | | |
| FAM154B | family with sequence similarity | up | 2.62 | 1.39 |
| | 154, member B | | | |
| SUPT3H | suppressor of Ty 3 homolog | up | 2.61 | 1.39 |
| | (S. cerevisiae) | | | |
| BAALC | brain and acute leukemia, | up | 2.61 | 1.38 |
| | cytoplasmic | | | |
| RIBC1 | RIB43A domain with coiled- | up | 2.59 | 1.37 |
| | coils 1 | | | |
| ZNF354C | zinc finger protein 354C | up | 2.59 | 1.37 |
| DIS3E2 | DIS3 like 3′-5′ exoribonuclease 2 | up | 2.58 | 1.37 |
| HIST1H2AA | histone cluster 1, H2aa | up | 2.55 | 1.35 |
| MYCNOS | MYCN opposite strand | up | 2.55 | 1.35 |
| PLA2G2D | phospholipase A2, group IID | up | 2.54 | 1.34 |
| ANKRD20A19P | ankyrin repeat domain 20 family, | up | 2.52 | 1.33 |
| | member A19, pseudogene | | | |
| SSPO | SCO-spondin | up | 2.52 | 1.33 |
| ARMCX4 | armadillo repeat containing, X- | up | 2.51 | 1.33 |
| | linked 4 | | | |
| SNAR-G1 | small ILF3/NF90-associated | up | 2.51 | 1.33 |
| | RNA G1 | | | |
| MYLK3 | myosin light chain kinase 3 | up | 2.50 | 1.32 |
| NPAS3 | neuronal PAS domain protein 3 | up | 2.49 | 1.31 |
| HS1BP3-IT1 | HS1BP3 intronic transcript 1 | up | 2.49 | 1.31 |
| | (non-protein coding) | | | |
| DTX3 | deltex 3, E3 ubiquitin ligase | up | 2.48 | 1.31 |
| PGM5P4-AS1 | PGM5P4 antisense RNA 1 | up | 2.46 | 1.30 |
| SNORA14B | small nucleolar RNA, H/ACA | up | 2.46 | 1.30 |
| | box 14B | | | |
| SLC39A2 | solute carrier family 39 (zinc | up | 2.46 | 1.30 |
| | transporter), member 2 | | | |
| ZNF287 | zinc finger protein 287 | up | 2.45 | 1.29 |
| GS1-259H13.2 | transmembrane protein 225-like | up | 2.45 | 1.29 |
| MRGPRG | MAS-related GPR, member G | up | 2.45 | 1.29 |
| LRRC17 | leucine rich repeat containing 17 | up | 2.45 | 1.29 |
| LANCL2 | LanC lantibiotic synthetase | up | 2.44 | 1.29 |
| | component C-like 2 (bacterial) | | | |
| ANKEF1 | ankyrin repeat and EF-hand | up | 2.44 | 1.29 |
| | domain containing 1 | | | |
| PBX2 | pre-B-cell leukemia homeobox 2 | up | 2.43 | 1.28 |
| CCDC121 | coiled-coil domain containing | up | 2.42 | 1.28 |
| | 121 | | | |
| CRYBA2 | crystallin, beta A2 | up | 2.42 | 1.28 |
| TMPO-AS1 | TMPO antisense RNA 1 | up | 2.42 | 1.28 |
| ACSBG1 | acyl-CoA synthetase bubblegum | up | 2.41 | 1.27 |
| | family member 1 | | | |
| MR1 | major histocompatibility | up | 2.41 | 1.27 |
| | complex, class I-related | | | |
| PPIP5K1 | diphosphoinositol | up | 2.41 | 1.27 |
| | pentakisphosphate kinase 1 | | | |
| TRIM6 | tripartite motif containing 6 | up | 2.41 | 1.27 |
| CCDC121 | coiled-coil domain containing | up | 2.41 | 1.27 |
| | 121 | | | |
| TUSC5 | tumor suppressor candidate 5 | up | 2.40 | 1.26 |
| XPO5 | exportin 5 | up | 2.39 | 1.26 |
| SPDYE8P | speedy/RINGO cell cycle | up | 2.39 | 1.26 |
| | regulator family member E8, | | | |
| | pseudogene | | | |
| CSRNP3 | cysteine-serine-rich nuclear | up | 2.39 | 1.26 |
| | protein 3 | | | |
| RBM33 | RNA binding motif protein 33 | up | 2.37 | 1.25 |
| B3GNT7 | UDP-GlcNAc:betaGal beta-1,3- | up | 2.37 | 1.24 |
| | N-acetylglucosaminyltransferase | | | |
| | 7 | | | |
| NME9 | NME/NM23 family member 9 | up | 2.36 | 1.24 |
| CYP4V2 | cytochrome P450, family 4, | up | 2.36 | 1.24 |
| | subfamily V, polypeptide 2 | | | |
| CDSN | corneodesmosin | up | 2.36 | 1.24 |
| COL20A1 | collagen, type XX, alpha 1 | up | 2.36 | 1.24 |
| ZNF192P1 | zinc finger protein 192 | up | 2.35 | 1.23 |
| | pseudogene 1 | | | |
| HERC6 | HECT and RLD domain | up | 2.35 | 1.23 |
| | containing E3 ubiquitin protein | | | |
| | ligase family member 6 | | | |
| SNORD25 | small nucleolar RNA, C/D box | up | 2.35 | 1.23 |
| | 25 | | | |
| FRS3 | fibroblast growth factor receptor | up | 2.35 | 1.23 |
| | substrate 3 | | | |
| RGS5 | regulator of G-protein signaling | up | 2.33 | 1.22 |
| | 5 | | | |
| RMST | rhabdomyosarcoma 2 associated | up | 2.33 | 1.22 |
| | transcript (non-protein coding) | | | |
| GAS2L2 | growth arrest-specific 2 like 2 | up | 2.33 | 1.22 |
| SGPP1 | sphingosine-1-phosphate | up | 2.32 | 1.21 |
| | phosphatase 1 | | | |
| NIPSNAP3B | nipsnap homolog 3B (C. elegans) | up | 2.31 | 1.21 |
| TAB3 | TGF-beta activated kinase | up | 2.31 | 1.21 |
| | 1/MAP3K7 binding protein 3 | | | |
| RASSF10 | Ras association (RalGDS/AF-6) | up | 2.31 | 1.21 |
| | domain family (N-terminal) | | | |
| | member 10 | | | |
| PPIL6 | peptidylprolyl isomerase | up | 2.31 | 1.20 |
| | (cyclophilin)-like 6 | | | |
| MPP4 | membrane protein, palmitoylated | up | 2.30 | 1.20 |
| | 4 (MAGUK p55 subfamily | | | |
| | member 4) | | | |
| ZNF37A | zinc finger protein 37A | up | 2.30 | 1.20 |
| ATG10 | autophagy related 10 | up | 2.29 | 1.20 |
| FPR3 | formyl peptide receptor 3 | up | 2.28 | 1.19 |
| PDP2 | pyruvate dehyrogenase | up | 2.27 | 1.18 |
| | phosphatase catalytic subunit 2 | | | |
| ZFAND4 | zinc finger, AN1-type domain 4 | up | 2.27 | 1.18 |
| RHOT1 | ras homolog family member T1 | up | 2.26 | 1.18 |
| NKAPP1 | NFKB activating protein | up | 2.26 | 1.18 |
| | pseudogene 1 | | | |
| OPTC | opticin | up | 2.26 | 1.18 |
| FLT4 | fms-related tyrosine kinase 4 | up | 2.26 | 1.18 |
| OR2AG1 | olfactory receptor, family 2, | up | 2.26 | 1.17 |
| | subfamily AG, member 1 | | | |
| | (gene/pseudogene) | | | |
| C9orf72 | chromosome 9 open reading | up | 2.25 | 1.17 |
| | frame 72 | | | |
| PPIP5K1 | diphosphoinositol | up | 2.25 | 1.17 |
| | pentakisphosphate kinase 1 | | | |
| ASIC3 | acid sensing (proton gated) ion | up | 2.24 | 1.17 |
| | channel 3 | | | |
| PIK3CD | phosphatidylinositol-4,5- | up | 2.24 | 1.16 |
| | bisphosphate 3-kinase, catalytic | | | |
| | subunit delta | | | |
| FAAHP1 | fatty acid amide hydrolase | up | 2.24 | 1.16 |
| | pseudogene 1 | | | |
| ZNF641 | zinc finger protein 641 | up | 2.24 | 1.16 |
| SCN1B | sodium channel, voltage gated, | up | 2.24 | 1.16 |
| | type I beta subunit | | | |
| FCN1 | ficolin (collagen/fibrinogen | up | 2.22 | 1.15 |
| | domain containing) 1 | | | |
| HIST1H4G | histone cluster 1, H4g | up | 2.22 | 1.15 |
| CEBPA | CCAAT/enhancer binding | up | 2.22 | 1.15 |
| | protein (C/EBP), alpha | | | |
| ANKRD20A8P | ankyrin repeat domain 20 family, | up | 2.22 | 1.15 |
| | member A8, pseudogene | | | |
| MLYCD | malonyl-CoA decarboxylase | up | 2.22 | 1.15 |
| C5orf56 | chromosome 5 open reading | up | 2.22 | 1.15 |
| | frame 56 | | | |
| HERC6 | HECT and RLD domain | up | 2.22 | 1.15 |
| | containing E3 ubiquitin protein | | | |
| | ligase family member 6 | | | |
| PRKACB | protein kinase, cAMP-dependent, | up | 2.22 | 1.15 |
| | catalytic, beta | | | |
| SRP14-AS1 | SRP14 antisense RNA1 (head to | up | 2.22 | 1.15 |
| | head) | | | |
| C1RL | complement component 1, r | up | 2.21 | 1.15 |
| | subcomponent-like | | | |
| EMR2 | egf-like module containing, | up | 2.21 | 1.15 |
| | mucin-like, hormone receptor- | | | |
| | like 2 | | | |
| ATPAF1 | ATP synthase mitochondrial F1 | up | 2.21 | 1.15 |
| | complex assembly factor 1 | | | |
| ZNF222 | zinc finger protein 222 | up | 2.20 | 1.14 |
| SCGB1B2P | secretoglobin, family 1B, | up | 2.20 | 1.14 |
| | member 2, pseudogene | | | |
| CCDC113 | coiled-coil domain containing | up | 2.20 | 1.14 |
| | 113 | | | |
| C15orf48 | chromosome 15 open reading | up | 2.20 | 1.14 |
| | frame 48 | | | |
| ETV3 | ets variant 3 | up | 2.20 | 1.14 |
| SCP2 | sterol carrier protein 2 | up | 2.20 | 1.14 |
| MGC34796 | sepiapterin reductase (7,8- | up | 2.19 | 1.13 |
| | dihydrobiopterin:NADP+ | | | |
| | oxidoreductase) pseudogene | | | |
| RAP1GAP | RAP1 GTPase activating protein | up | 2.19 | 1.13 |
| MAGOHB | mago-nashi homolog B | up | 2.19 | 1.13 |
| | (Drosophila) | | | |
| ZBED4 | zinc finger, BED-type containing | up | 2.19 | 1.13 |
| | 4 | | | |
| FAM98B | family with sequence similarity | up | 2.18 | 1.13 |
| | 98, member B | | | |
| EPHB1 | EPH receptor B1 | up | 2.18 | 1.13 |
| POM121 | POM121 transmembrane | up | 2.18 | 1.12 |
| | nucleoporin | | | |
| SNX21 | sorting nexin family member 21 | up | 2.18 | 1.12 |
| OR6Y1 | olfactory receptor, family 6, | up | 2.18 | 1.12 |
| | subfamily Y, member 1 | | | |
| LMBRD2 | LMBR1 domain containing 2 | up | 2.18 | 1.12 |
| FLVCR2 | feline leukemia virus subgroup C | up | 2.17 | 1.12 |
| | cellular receptor family, member | | | |
| | 2 | | | |
| LRRC15 | leucine rich repeat containing 15 | up | 2.17 | 1.12 |
| TGFBR3 | transforming growth factor, beta | up | 2.17 | 1.12 |
| | receptor III | | | |
| NXT2 | nuclear transport factor 2-like | up | 2.17 | 1.11 |
| | export factor 2 | | | |
| HCRP1 | hepatocellular carcinoma-related | up | 2.17 | 1.11 |
| | HCRP1 | | | |
| HEPACAM | hepatic and glial cell adhesion | up | 2.16 | 1.11 |
| | molecule | | | |
| OBSCN | obscurin, cytoskeletal calmodulin | up | 2.16 | 1.11 |
| | and titin-interacting RhoGEF | | | |
| SQSTM1 | sequestosome 1 | up | 2.16 | 1.11 |
| LGALS8 | lectin, galactoside-binding, | up | 2.15 | 1.11 |
| | soluble, 8 | | | |
| RHBDL1 | rhomboid, veinlet-like 1 | up | 2.15 | 1.11 |
| | (Drosophila) | | | |
| KIF21B | kinesin family member 21B | up | 2.15 | 1.10 |
| CEP170 | centrosomal protein 170 kDa | up | 2.15 | 1.10 |
| CYP51A1-AS1 | CYP51A1 antisense RNA 1 | up | 2.15 | 1.10 |
| LIMD1 | LIM domains containing 1 | up | 2.15 | 1.10 |
| ANKRD53 | ankyrin repeat domain 53 | up | 2.14 | 1.10 |
| INGX | inhibitor of growth family, X- | up | 2.14 | 1.10 |
| | linked, pseudogene | | | |
| CDH15 | cadherin 15, type 1, M-cadherin | up | 2.14 | 1.10 |
| | (myotubule) | | | |
| APCS | amyloid P component, serum | up | 2.14 | 1.10 |
| SYN2 | synapsin II | up | 2.14 | 1.10 |
| SMKR1 | small lysine-rich protein 1 | up | 2.13 | 1.09 |
| KCNK7 | potassium channel, two pore | up | 2.13 | 1.09 |
| | domain subfamily K, member 7 | | | |
| TEX28 | testis expressed 28 | up | 2.13 | 1.09 |
| DYNLRB2 | dynein, light chain, roadblock- | up | 2.13 | 1.09 |
| | type 2 | | | |
| CNNM2 | cyclin and CBS domain divalent | up | 2.13 | 1.09 |
| | metal cation transport mediator 2 | | | |
| RSG1 | REM2 and RAB-like small | up | 2.13 | 1.09 |
| | GTPase 1 | | | |
| FRMD8 | FERM domain containing 8 | up | 2.13 | 1.09 |
| STK33 | serine/threonine kinase 33 | up | 2.13 | 1.09 |
| OPLAH | 5-oxoprolinase (ATP- | up | 2.13 | 1.09 |
| | hydrolysing) | | | |
| EFHC1 | EF-hand domain (C-terminal) | up | 2.13 | 1.09 |
| | containing 1 | | | |
| C16orf47 | chromosome 16 open reading | up | 2.13 | 1.09 |
| | frame 47 | | | |
| GSTO2 | glutathione S-transferase omega | up | 2.12 | 1.09 |
| | 2 | | | |
| PTPLB | protein tyrosine phosphatase-like | up | 2.12 | 1.09 |
| | (proline instead of catalytic | | | |
| | arginine), member b | | | |
| HIST1H2APS1 | histone cluster 1, H2a, | up | 2.12 | 1.09 |
| | pseudogene 1 | | | |
| OR10W1 | olfactory receptor, family 10, | up | 2.12 | 1.08 |
| | subfamily W, member 1 | | | |
| PEX5 | peroxisomal biogenesis factor 5 | up | 2.12 | 1.08 |
| ASS1 | argininosuccinate synthase 1 | up | 2.11 | 1.08 |
| LRRC16B | leucine rich repeat containing | up | 2.11 | 1.08 |
| | 16B | | | |
| CWC15 | CWC15 spliceosome-associated | up | 2.11 | 1.08 |
| | protein | | | |
| SUV420H1 | suppressor of variegation 4-20 | up | 2.11 | 1.08 |
| | homolog 1 (Drosophila) | | | |
| TET3 | tet methylcytosine dioxygenase 3 | up | 2.11 | 1.07 |
| POLD2 | polymerase (DNA directed), | up | 2.10 | 1.07 |
| | delta 2, accessory subunit | | | |
| CCDC169 | coiled-coil domain containing | up | 2.10 | 1.07 |
| | 169 | | | |
| QRICH2 | glutamine rich 2 | up | 2.10 | 1.07 |
| SLC9A5 | solute carrier family 9, subfamily | up | 2.10 | 1.07 |
| | A (NHE5, cation proton | | | |
| | antiporter 5), member 5 | | | |
| CFAP69 | cilia and flagella associated | up | 2.10 | 1.07 |
| | protein 69 | | | |
| HSF1 | heat shock transcription factor 1 | up | 2.09 | 1.07 |
| ACTB | actin, beta | up | 2.09 | 1.06 |
| MYPOP | Myb-related transcription factor, | up | 2.09 | 1.06 |
| | partner of profilin | | | |
| HOXA11 | homeobox A11 | up | 2.08 | 1.06 |
| GNG12-AS1 | GNG12 antisense RNA 1 | up | 2.08 | 1.06 |
| TAPBP | TAP binding protein (tapasin) | up | 2.08 | 1.06 |
| DLEU2L | deleted in lymphocytic leukemia | up | 2.08 | 1.06 |
| | 2-like | | | |
| TESC | tescalcin | up | 2.08 | 1.06 |
| ITGA8 | integrin, alpha 8 | up | 2.08 | 1.05 |
| PPARGC1B | peroxisome proliferator-activated | up | 2.08 | 1.05 |
| | receptor gamma, coactivator 1 | | | |
| | beta | | | |
| FAM26E | family with sequence similarity | up | 2.08 | 1.05 |
| | 26, member E | | | |
| OR2A9P | olfactory receptor, family 2, | up | 2.08 | 1.05 |
| | subfamily A, member 9 | | | |
| | pseudogene | | | |
| WHAMMP3 | WAS protein homolog associated | up | 2.07 | 1.05 |
| | with actin, golgi membranes and | | | |
| | microtubules pseudogene 3 | | | |
| BTNL10 | butyrophilin-like 10 | up | 2.07 | 1.05 |
| PADI6 | peptidyl arginine deiminase, type | up | 2.07 | 1.05 |
| | VI | | | |
| TMEM86A | transmembrane protein 86A | up | 2.06 | 1.04 |
| PARK2 | parkin RBR E3 ubiquitin protein | up | 2.06 | 1.04 |
| | ligase | | | |
| MAPT | microtubule-associated protein | up | 2.06 | 1.04 |
| | tau | | | |
| MAGEC1 | melanoma antigen family C, 1 | up | 2.06 | 1.04 |
| PYCR1 | pyrroline-5-carboxylate | up | 2.06 | 1.04 |
| | reductase 1 | | | |
| CTBP1-AS2 | CTBP1 antisense RNA 2 (head | up | 2.05 | 1.04 |
| | to head) | | | |
| GGCT | gamma-glutamylcyclotransferase | up | 2.05 | 1.04 |
| FAM25A | family with sequence similarity | up | 2.05 | 1.03 |
| | 25, member A | | | |
| PTCH1 | patched 1 | up | 2.05 | 1.03 |
| ALDH5A1 | aldehyde dehydrogenase 5 | up | 2.05 | 1.03 |
| | family, member A1 | | | |
| CCDC68 | coiled-coil domain containing 68 | up | 2.05 | 1.03 |
| DAB2IP | DAB2 interacting protein | up | 2.05 | 1.03 |
| ATAD3C | ATPase family, AAA domain | up | 2.05 | 1.03 |
| | containing 3C | | | |
| MARVELD2 | MARVEL domain containing 2 | up | 2.04 | 1.03 |
| KLC4 | kinesin light chain 4 | up | 2.04 | 1.03 |
| SREK1IP1 | SREK1-interacting protein 1 | up | 2.04 | 1.03 |
| CHAC2 | ChaC, cation transport regulator | up | 2.04 | 1.03 |
| | homolog 2 (E. coli) | | | |
| HDHD3 | haloacid dehalogenase-like | up | 2.04 | 1.03 |
| | hydrolase domain containing 3 | | | |
| ELMO2 | engulfment and cell motility 2 | up | 2.04 | 1.03 |
| TMSB4Y | thymosin beta 4, Y-linked | up | 2.04 | 1.03 |
| CYP2B6 | cytochrome P450, family 2, | up | 2.03 | 1.02 |
| | subfamily B, polypeptide 6 | | | |
| EDNRB | endothelin receptor type B | up | 2.03 | 1.02 |
| ZNF660 | zinc finger protein 660 | up | 2.03 | 1.02 |
| SLC2A3 | solute carrier family 2 (facilitated | up | 2.03 | 1.02 |
| | glucose transporter), member 3 | | | |
| ZNF749 | zinc finger protein 749 | up | 2.03 | 1.02 |
| C2orf27A | chromosome 2 open reading | up | 2.03 | 1.02 |
| | frame 27A | | | |
| STRADA | STE20-related kinase adaptor | up | 2.03 | 1.02 |
| | alpha | | | |
| MIR99AHG | mir-99a-let-7c cluster host gene | up | 2.03 | 1.02 |
| | (non-protein coding) | | | |
| ZNF671 | zinc finger protein 671 | up | 2.02 | 1.02 |
| CEP44 | centrosomal protein 44 kDa | up | 2.02 | 1.02 |
| VPS9D1-AS1 | VPS9D1 antisense RNA 1 | up | 2.02 | 1.01 |
| SMAD1-AS1 | SMAD1 antisense RNA 1 | up | 2.02 | 1.01 |
| UBE3C | ubiquitin protein ligase E3C | up | 2.02 | 1.01 |
| PRSS33 | protease, serine, 33 | up | 2.02 | 1.01 |
| TTC28 | tetratricopeptide repeat domain | up | 2.02 | 1.01 |
| | 28 | | | |
| RASGRF1 | Ras protein-specific guanine | up | 2.01 | 1.01 |
| | nucleotide-releasing factor 1 | | | |
| CFH | complement factor H | up | 2.01 | 1.01 |
| GNG12-AS1 | GNG12 antisense RNA 1 | up | 2.01 | 1.01 |
| GUSBP5 | glucuronidase, beta pseudogene | up | 2.01 | 1.01 |
| | 5 | | | |
| MMRN2 | multimerin 2 | up | 2.00 | 1.00 |
| HIF1A | hypoxia inducible factor 1, alpha | up | 2.00 | 1.00 |
| | subunit (basic helix-loop-helix | | | |
| | transcription factor) | | | |
| ZNF271P | zinc finger protein 271, | up | 2.00 | 1.00 |
| | pseudogene | | | |
| DBF4B | DBF4 zinc finger B | up | 2.00 | 1.00 |
| NRCAM | neuronal cell adhesion molecule | up | 2.00 | 1.00 |
| MKS1 | Meckel syndrome, type 1 | up | 2.00 | 1.00 |
| GAREML | GRB2 associated, regulator of | down | −6.04 | −2.59 |
| | MAPK1-like | | | |
| SNORA2A | small nucleolar RNA, H/ACA | down | −6.04 | −2.59 |
| | box 2A | | | |
| MUC3A | mucin 3A, cell surface associated | down | −6.04 | −2.59 |
| MAP3K10 | mitogen-activated protein kinase | down | −6.12 | −2.61 |
| | kinase kinase 10 | | | |
| SASS6 | spindle assembly 6 homolog | down | −6.15 | −2.62 |
| | (C. elegans) | | | |
| BANP | BTG3 associated nuclear protein | down | −6.16 | −2.62 |
| TSPAN15 | tetraspanin 15 | down | −6.23 | −2.64 |
| SLC25A17 | solute carrier family 25 | down | −6.27 | −2.65 |
| | (mitochondrial carrier; | | | |
| | peroxisomal membrane protein, | | | |
| | 34 kDa), member 17 | | | |
| SLC5A1 | solute carrier family 5 | down | −6.36 | −2.67 |
| | (sodium/glucose co transporter), | | | |
| | member 1 | | | |
| HYOU1 | hypoxia up-regulated 1 | down | −6.37 | −2.67 |
| MYH3 | myosin, heavy chain 3, skeletal | down | −6.38 | −2.67 |
| | muscle, embryonic | | | |
| FCRL5 | Fc receptor-like 5 | down | −6.39 | −2.67 |
| DIS3L2 | DIS3 like 3′-5′ exoribonuclease 2 | down | −6.43 | −2.69 |
| SUCNR1 | succinate receptor 1 | down | −6.44 | −2.69 |
| NPBWR1 | neuropeptides B/W receptor 1 | down | −6.53 | −2.71 |
| MDM2 | MDM2 proto-oncogene, E3 | down | −6.56 | −2.71 |
| | ubiquitin protein ligase | | | |
| HIST1H2AC | histone cluster 1, H2ac | down | −6.56 | −2.71 |
| KLRC1 | killer cell lectin-like receptor | down | −6.58 | −2.72 |
| | subfamily C, member 1 | | | |
| CDCA7L | cell division cycle associated 7- | down | −6.59 | −2.72 |
| | like | | | |
| RNA28S5 | RNA, 28S ribosomal 5 | down | −6.65 | −2.73 |
| MAGT1 | magnesium transporter 1 | down | −6.68 | −2.74 |
| HYDIN | HYDIN, axonemal central pair | down | −6.68 | −2.74 |
| | apparatus protein | | | |
| FAM96B | family with sequence similarity | down | −6.70 | −2.74 |
| | 96, member B | | | |
| FPGT | fucose-1-phosphate | down | −6.70 | −2.74 |
| | guanylyltransferase | | | |
| USP45 | ubiquitin specific peptidase 45 | down | −6.72 | −2.75 |
| FAM87A | family with sequence similarity | down | −6.72 | −2.75 |
| | 87, member A | | | |
| LMO7DN | LMO7 downstream neighbor | down | −6.78 | −2.76 |
| LACC1 | laccase (multicopper | down | −6.80 | −2.77 |
| | oxidoreductase) domain | | | |
| | containing 1 | | | |
| BAIAP2-AS1 | BAIAP2 antisense RNA 1 (head | down | −6.87 | −2.78 |
| | to head) | | | |
| CACNA1I | calcium channel, voltage- | down | −6.88 | −2.78 |
| | dependent, T type, alpha 11 | | | |
| | subunit | | | |
| MB21D2 | Mab-21 domain containing 2 | down | −6.91 | −2.79 |
| AMOTL1 | angiomotin like 1 | down | −7.01 | −2.81 |
| PALLD | palladin, cytoskeletal associated | down | −7.02 | −2.81 |
| | protein | | | |
| HTT | huntingtin | down | −7.09 | −2.83 |
| CASC8 | cancer susceptibility candidate 8 | down | −7.14 | −2.84 |
| | (non-protein coding) | | | |
| C11orf68 | chromosome 11 open reading | down | −7.20 | −2.85 |
| | frame 68 | | | |
| ZNF778 | zinc finger protein 778 | down | −7.25 | −2.86 |
| FUT7 | fucosyltransferase 7 (alpha (1,3) | down | −7.26 | −2.86 |
| | fucosyltransferase) | | | |
| CT55 | cancer/testis antigen 55 | down | −7.29 | −2.87 |
| ULK4 | unc-51 like kinase 4 | down | −7.51 | −2.91 |
| EIF2AK1 | eukaryotic translation initiation | down | −7.53 | −2.91 |
| | factor 2-alpha kinase 1 | | | |
| STON1 | stonin 1 | down | −7.56 | −2.92 |
| NOX1 | NADPH oxidase 1 | down | −7.63 | −2.93 |
| METAP1D | methionyl aminopeptidase type | down | −7.67 | −2.94 |
| | 1D (mitochondrial) | | | |
| FAM120B | family with sequence similarity | down | −7.68 | −2.94 |
| | 120B | | | |
| IMPG1 | interphotoreceptor matrix | down | −7.76 | −2.96 |
| | proteoglycan 1 | | | |
| PBX2 | pre-B-cell leukemia homeobox 2 | down | −7.77 | −2.96 |
| POLR2F | polymerase (RNA) II (DNA | down | −7.81 | −2.97 |
| | directed) polypeptide F | | | |
| GLI2 | GLI family zinc finger 2 | down | −7.93 | −2.99 |
| MRPL28 | mitochondrial ribosomal protein | down | −7.94 | −2.99 |
| | L28 | | | |
| STEAP3 | STEAP family member 3, | down | −8.08 | −3.01 |
| | metalloreductase | | | |
| USP2 | ubiquitin specific peptidase 2 | down | −8.09 | −3.02 |
| SEL1L | sel-1 suppressor of lin-12-like | down | −8.21 | −3.04 |
| | (C. elegans) | | | |
| PCDH17 | protocadherin 17 | down | −8.25 | −3.04 |
| ASB1 | ankyrin repeat and SOCS box | down | −8.30 | −3.05 |
| | containing 1 | | | |
| PER2 | period circadian clock 2 | down | −8.39 | −3.07 |
| KRT8P12 | keratin 8 pseudogene 12 | down | −8.45 | −3.08 |
| SYNJ2-IT1 | SYNJ2 intronic transcript 1 (non- | down | −8.46 | −3.08 |
| | protein coding) | | | |
| ADCK5 | aarF domain containing kinase 5 | down | −8.46 | −3.08 |
| ATP2B1 | ATPase, Ca++ transporting, | down | −8.49 | −3.09 |
| | plasma membrane 1 | | | |
| GEMIN2 | gem (nuclear organelle) | down | −8.50 | −3.09 |
| | associated protein 2 | | | |
| EIF4B | eukaryotic translation initiation | down | −8.54 | −3.09 |
| | factor 4B | | | |
| IFNK | interferon, kappa | down | −8.63 | −3.11 |
| SPATS2L | spermatogenesis associated, | down | −8.63 | −3.11 |
| | serine-rich 2-like | | | |
| SUCLG2-AS1 | SUCLG2 antisense RNA 1 (head | down | −8.67 | −3.12 |
| | to head) | | | |
| OTUD7B | OTU deubiquitinase 7B | down | −8.67 | −3.12 |
| HAPLN2 | hyaluronan and proteoglycan link | down | −8.69 | −3.12 |
| | protein 2 | | | |
| TRMT13 | tRNA methyltransferase 13 | down | −8.72 | −3.12 |
| | homolog (S. cerevisiae) | | | |
| PRR27 | proline rich 27 | down | −8.73 | −3.13 |
| RCSD1 | RCSD domain containing 1 | down | −8.81 | −3.14 |
| FBXO24 | F-box protein 24 | down | −8.99 | −3.17 |
| SYK | spleen tyrosine kinase | down | −9.02 | −3.17 |
| TMEM129 | transmembrane protein 129, E3 | down | −9.04 | −3.18 |
| | ubiquitin protein ligase | | | |
| CDRT1 | CMT1A duplicated region | down | −9.05 | −3.18 |
| | transcript 1 | | | |
| CDK5RAP3 | CDK5 regulatory subunit | down | −9.11 | −3.19 |
| | associated protein 3 | | | |
| OPTN | optineurin | down | −9.13 | −3.19 |
| CYLC1 | cylicin, basic protein of sperm | down | −9.21 | −3.20 |
| | head cytoskeleton 1 | | | |
| CAPN3 | calpain 3, (p94) | down | −9.35 | −3.23 |
| LARGE-AS1 | LARGE antisense RNA 1 | down | −9.36 | −3.23 |
| CXorf40B | chromosome X open reading | down | −9.44 | −3.24 |
| | frame 40B | | | |
| NINL | ninein-like | down | −9.44 | −3.24 |
| ETV5 | ets variant 5 | down | −9.45 | −3.24 |
| ZNF585A | zinc finger protein 585A | down | −9.59 | −3.26 |
| ARHGAP5-AS1 | ARHGAP5 antisense RNA 1 | down | −9.63 | −3.27 |
| | (head to head) | | | |
| ZNF594 | zinc finger protein 594 | down | −9.65 | −3.27 |
| ALS2 | amyotrophic lateral sclerosis 2 | down | −9.76 | −3.29 |
| | (juvenile) | | | |
| SNX10 | sorting nexin 10 | down | −9.76 | −3.29 |
| LGI2 | leucine-rich repeat LGI family, | down | −9.80 | −3.29 |
| | member 2 | | | |
| BTG3 | BTG family, member 3 | down | −9.93 | −3.31 |
| LCE2A | late cornified envelope 2A | down | −10.02 | −3.32 |
| CC2D1B | coiled-coil and C2 domain | down | −10.29 | −3.36 |
| | containing 1B | | | |
| DIP2A | DIP2 disco-interacting protein 2 | down | −10.43 | −3.38 |
| | homolog A (Drosophila) | | | |
| RFNG | RFNG O-fucosylpeptide 3-beta- | down | −10.55 | −3.40 |
| | N-acetylglucosaminyltransferase | | | |
| SLN | sarcolipin | down | −10.56 | −3.40 |
| NUP98 | nucleoporin 98 kDa | down | −10.65 | −3.41 |
| NSA2 | NSA2 ribosome biogenesis | down | −10.70 | −3.42 |
| | homolog (S. cerevisiae) | | | |
| KIF1C | kinesin family member 1C | down | −10.72 | −3.42 |
| ZIC3 | Zic family member 3 | down | −11.34 | −3.50 |
| MFAP3 | microfibrillar-associated protein | down | −11.37 | −3.51 |
| | 3 | | | |
| GNA15 | guanine nucleotide binding | down | −11.46 | −3.52 |
| | protein (G protein), alpha 15 (Gq | | | |
| | class) | | | |
| MAGEA6 | melanoma antigen family A, 6 | down | −11.60 | −3.54 |
| MCAT | malonyl CoA:ACP | down | −11.65 | −3.54 |
| | acyltransferase (mitochondrial) | | | |
| MCFD2 | multiple coagulation factor | down | −12.07 | −3.59 |
| | deficiency 2 | | | |
| SNORD15A | small nucleolar RNA, C/D box | down | −12.11 | −3.60 |
| | 15A | | | |
| KCNE5 | potassium channel, voltage gated | down | −12.25 | −3.61 |
| | subfamily E regulatory beta | | | |
| | subunit 5 | | | |
| MAGI2-AS3 | MAGI2 antisense RNA 3 | down | −12.33 | −3.62 |
| NAA60 | N(alpha)-acetyltransferase 60, | down | −12.34 | −3.63 |
| | NatF catalytic subunit | | | |
| GABBR1 | gamma-aminobutyric acid | down | −12.73 | −3.67 |
| | (GABA) B receptor, 1 | | | |
| TGFB1I1 | transforming growth factor beta | down | −12.84 | −3.68 |
| | 1 induced transcript 1 | | | |
| LPAR6 | lysophosphatidic acid receptor 6 | down | −13.11 | −3.71 |
| RNASE7 | ribonuclease, RNase A family, 7 | down | −13.47 | −3.75 |
| MAFG | v-maf avian musculoaponeurotic | down | −13.80 | −3.79 |
| | fibrosarcoma oncogene homolog | | | |
| | G | | | |
| COL6A4P1 | collagen, type VI, alpha 4 | down | −15.64 | −3.97 |
| | pseudogene 1 | | | |
| NAV2-AS5 | NAV2 antisense RNA 5 | down | −15.83 | −3.98 |
| OR52B2 | olfactory receptor, family 52, | down | −17.06 | −4.09 |
| | subfamily B, member 2 | | | |
| SLC19A1 | solute carrier family 19 (folate | down | −17.20 | −4.10 |
| | transporter), member 1 | | | |
| HIST1H2BM | histone cluster 1, H2bm | down | −17.25 | −4.11 |
| LRRC17 | leucine rich repeat containing 17 | down | −17.46 | −4.13 |
| TNFRSF10C | tumor necrosis factor receptor | down | −17.69 | −4.15 |
| | superfamily, member 10c, decoy | | | |
| | without an intracellular domain | | | |
| ARHGEF7 | Rho guanine nucleotide | down | −18.02 | −4.17 |
| | exchange factor (GEF) 7 | | | |
| OR4C6 | olfactory receptor, family 4, | down | −18.80 | −4.23 |
| | subfamily C, member 6 | | | |
| CCDC158 | coiled-coil domain containing | down | −18.86 | −4.24 |
| | 158 | | | |
| RNFT2 | ring finger protein, | down | −19.57 | −4.29 |
| | transmembrane 2 | | | |
| MAPKAPK5- | MAPKAPK5 antisense RNA 1 | down | −19.79 | −4.31 |
| AS1 | | | | |
| MYO15B | myosin XVB pseudogene | down | −20.06 | −4.33 |
| ANKRD54 | ankyrin repeat domain 54 | down | −20.15 | −4.33 |
| CLCA4 | chloride channel accessory 4 | down | −20.19 | −4.34 |
| DNAJB1 | DnaJ (Hsp40) homolog, | down | −20.40 | −4.35 |
| | subfamily B, member 1 | | | |
| REEP3 | receptor accessory protein 3 | down | −21.78 | −4.44 |
| PGBD2 | piggyBac transposable element | down | −21.84 | −4.45 |
| | derived 2 | | | |
| CENPV | centromere protein V | down | −22.45 | −4.49 |
| CEP83 | centrosomal protein 83 kDa | down | −22.45 | −4.49 |
| TMEM200C | transmembrane protein 200C | down | −24.02 | −4.59 |
| RALBP1 | ralA binding protein 1 | down | −24.51 | −4.62 |
| DHFRL1 | dihydrofolate reductase-like 1 | down | −24.69 | −4.63 |
| TDRKH | tudor and KH domain containing | down | −25.59 | −4.68 |
| ALG3 | ALG3, alpha-1,3- | down | −26.75 | −4.74 |
| | mannosyltransferase | | | |
| CKS1B | CDC28 protein kinase regulatory | down | −27.57 | −4.78 |
| | subunit 1B | | | |
| KIAA1841 | KIAA1841 | down | −27.85 | −4.80 |
| BHLHE41 | basic helix-loop-helix family, | down | −28.68 | −4.84 |
| | member e41 | | | |
| DNAJC3 | DnaJ (Hsp40) homolog, | down | −29.22 | −4.87 |
| | subfamily C, member 3 | | | |
| C3orf17 | chromosome 3 open reading | down | −29.27 | −4.87 |
| | frame 17 | | | |
| GTF2H5 | general transcription factor IIH, | down | −29.36 | −4.88 |
| | polypeptide 5 | | | |
| PTGR1 | prostaglandin reductase 1 | down | −30.46 | −4.93 |
| JUN | jun proto-oncogene | down | −31.81 | −4.99 |
| TTC28-AS1 | TTC28 antisense RNA 1 | down | −32.23 | −5.01 |
| CHST2 | carbohydrate (N- | down | −32.67 | −5.03 |
| | acetylglucosamine-6-O) | | | |
| | sulfotransferase 2 | | | |
| KIF22 | kinesin family member 22 | down | −34.17 | −5.09 |
| CRHR1-IT1 | CRHR1 intronic transcript 1 | down | −34.51 | −5.11 |
| | (non-protein coding) | | | |
| ZNF746 | zinc finger protein 746 | down | −34.76 | −5.12 |
| ZNF658 | zinc finger protein 658 | down | −36.25 | −5.18 |
| ALS2 | amyotrophic lateral sclerosis 2 | down | −38.73 | −5.28 |
| | (juvenile) | | | |
| MTHFD1L | methylenetetrahydrofolate | down | −39.85 | −5.32 |
| | dehydrogenase (NADP+ | | | |
| | dependent) 1-like | | | |
| AMACR | alpha-methylacyl-CoA racemase | down | −41.76 | −5.38 |
| COMMD7 | COMM domain containing 7 | down | −45.82 | −5.52 |
| FIP1L1 | factor interacting with PAPOLA | down | −47.06 | −5.56 |
| | and CPSF1 | | | |
| PARP9 | poly (ADP-ribose) polymerase | down | −50.35 | −5.65 |
| | family, member 9 | | | |
| HIPK2 | homeodomain interacting protein | down | −50.40 | −5.66 |
| | kinase 2 | | | |
| AFF4 | AF4/FMR2 family, member 4 | down | −50.87 | −5.67 |
| EPB41L4A | erythrocyte membrane protein | down | −52.62 | −5.72 |
| | band 4.1 like 4A | | | |
| ZNF658 | zinc finger protein 658 | down | −54.60 | −5.77 |
| EDARADD | EDAR-associated death domain | down | −60.04 | −5.91 |
| PHB | prohibitin | down | −62.21 | −5.96 |
| ZNF532 | zinc finger protein 532 | down | −67.26 | −6.07 |
| HOXD-AS2 | HOXD cluster antisense RNA 2 | down | −69.02 | −6.11 |
| DSE | dermatan sulfate epimerase | down | −72.55 | −6.18 |
| NR2C1 | nuclear receptor subfamily 2, | down | −75.82 | −6.24 |
| | group C, member 1 | | | |
| ACSF3 | acyl-CoA synthetase family | down | −76.12 | −6.25 |
| | member 3 | | | |
| SNORA11C | small nucleolar RNA, H/ACA | down | −76.18 | −6.25 |
| | box 11C | | | |
| TMEM144 | transmembrane protein 144 | down | −77.84 | −6.28 |
| CD2AP | CD2-associated protein | down | −83.67 | −6.39 |
| CHML | choroideremia-like (Rab escort | down | −90.20 | −6.50 |
| | protein 2) | | | |
| RNF2 | ring finger protein 2 | down | −106.09 | −6.73 |
| HIST1H2BB | histone cluster 1, H2bb | down | −108.05 | −6.76 |
| INTS1 | integrator complex subunit 1 | down | −112.31 | −6.81 |
| IFT172 | intraflagellar transport 172 | down | −118.33 | −6.89 |
| PPIA | peptidylprolyl isomerase A | down | −123.27 | −6.95 |
| | (cyclophilin A) | | | |
| SLC26A6 | solute carrier family 26 (anion | down | −125.10 | −6.97 |
| | exchanger), member 6 | | | |
| DOPEY1 | dopey family member 1 | down | −125.80 | −6.98 |
| SLC22A23 | solute carrier family 22, member | down | −131.70 | −7.04 |
| | 23 | | | |
| SH3BP4 | SH3-domain binding protein 4 | down | −132.76 | −7.05 |
| WDR13 | WD repeat domain 13 | down | −134.84 | −7.08 |
| CSNK1G3 | casein kinase 1, gamma 3 | down | −141.58 | −7.15 |
| ROM1 | retinal outer segment membrane | down | −145.30 | −7.18 |
| | protein 1 | | | |
| MCM4 | minichromosome maintenance | down | −148.04 | −7.21 |
| | complex component 4 | | | |
| HHIP-AS1 | HHIP antisense RNA 1 | down | −158.31 | −7.31 |
| MFAP3L | microfibrillar-associated protein | down | −162.61 | −7.35 |
| | 3-like | | | |
| ALG1 | ALG1, | down | −181.56 | −7.50 |
| | chitobiosyldiphosphodolichol | | | |
| | beta-mannosyltransferase | | | |
| TAPT1 | transmembrane anterior posterior | down | −183.15 | −7.52 |
| | transformation 1 | | | |
| AASDHPPT | aminoadipate-semialdehyde | down | −194.22 | −7.60 |
| | dehydrogenase- | | | |
| | phosphopantetheinyl transferase | | | |
| RHOU | ras homolog family member U | down | −207.00 | −7.69 |
| AP1G2 | adaptor-related protein complex | down | −208.91 | −7.71 |
| | 1, gamma 2 subunit | | | |
| HSPA4 | heat shock 70 kDa protein 4 | down | −222.32 | −7.80 |
| HERC2P2 | hect domain and RLD 2 | down | −257.78 | −8.01 |
| | pseudogene 2 | | | |
| PABPC1 | poly(A) binding protein, | down | −285.37 | −8.16 |
| | cytoplasmic 1 | | | |
| ANKRD17 | ankyrin repeat domain 17 | down | −292.05 | −8.19 |
| SERP1 | stress-associated endoplasmic | down | −302.29 | −8.24 |
| | reticulum protein 1 | | | |
| RALGAPB | Ral GTPase activating protein, | down | −322.52 | −8.33 |
| | beta subunit (non-catalytic) | | | |
| UBE2Q2P1 | ubiquitin-conjugating enzyme | down | −326.90 | −8.35 |
| | E2Q family member 2 | | | |
| | pseudogene 1 | | | |
| PLXNA1 | plexin A1 | down | −423.72 | −8.73 |
| ATXN2 | ataxin 2 | down | −427.53 | −8.74 |
| ANKRD40 | ankyrin repeat domain 40 | down | −644.04 | −9.33 |
| RECK | reversion-inducing-cysteine-rich | down | −655.93 | −9.36 |
| | protein with kazal motifs | | | |
| EIF4E2 | eukaryotic translation initiation | down | −682.42 | −9.41 |
| | factor 4E family member 2 | | | |
| CYC1 | cytochrome c-1 | down | −994.35 | −9.96 |

The above studies, including in vitro experiments and in vivo mouse models of lymphedema, illustrate the potential of therapeutic use of ADSC-EVs for lymphedema treatment. In vitro studies revealed that ADSC-EVs not only have angiogenesis activity, but also lymphangiogeneisis activity. In vivo local injection of ADSC-EVs in lymphedema legs contributed the reduction of enlarged circumference and the induction of capillary vessels and lymphatic vessels. Also, fibrosis of tissue was decreased in ADSC-EVs treatment. Furthermore, the results show that inducing angiogenesis and lymphangiogenesis simultaneously may lead to formation of vessels which expressed both vascular and lymphatic markers and may have function for draining lymphatic system to vascular system, which may work as drainage routes of accumulated fluids and lead to reduction of swelling in lymphedema animal models. Importantly, the results show that lymphedema may be treated with a local application of ADSC-EVs, which also may reduce inflammatory responses, thus decreasing the formation of fibrosis. Results also show that ADSC-EVs contain heterogeneous miRNAs including miR-199a-3p, miR-145-5p, miR-143-3p, miR-377-3p, miR-100-3p, miR-29a-3p, miR-495-3p, and miR-29c-3p which target MDM2, and miR-658, miR-493-3p, miR-184, and miR-27a-3p which target PHB, related to lymphangiogenesis and angiogenesis. MDM2 is a cellular oncoprotein encoded by a gene located on chromosome 12q13-14. MDM2 can suppress p53, a cancer suppressor gene (Duffy, et al., Semin Cancer Biol., S1044-579X(20):30160-7 (2020)). The inhibition of MDM2 exerts antiinflammatory effects and may lead to the treatment of autoimmune diseases and cancer (Ebrihim, et al., Histol Histopathol., 30(11):1271-82) (2015). Previous studies have shown that MDM2 negatively regulates the stability of hypoxiainducible factor-1 alpha (HIF1a) at the protein level (Shweta, et al., J Biol Chem., 289(33):22785-97 (2014)). The studies reported herein found that the expression level of HIF1a was elevated in LECs after the addition of ADSC-EVs. HIF1a is a transcription factor that controls the cellular response to hypoxia. HIF1a promotes the transcription of various proteins, such as VEGF, Erythropoietin (EPO) and glucose transporters, and plays a key role in lymphangiogenesis and angiogenesis. Thus, these results are consistent with the conclusions that reduced expression of MDM2 caused by eight lymphangiogenic miRNAs in ADSC-EVs led to increase in HIF1a expression in LECs, which caused angiogenesis and lymphangiogenesis.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of promoting generation or regeneration of the lymphatic system in a subject comprising administering the subject a composition comprising an effective amount of extracellular vesicles formed by mesenchymal stem cells (MSCs) to increase generation of the lymphatic system.

2. The method of claim 1, wherein the composition is cell-free.

3. The method of claim 1, wherein the extracellular vesicles are formed by a method comprising culturing MSCs to produce media conditioned with the extracellular vesicles.

4. The method of claim 3, wherein the method further comprises separating extracellular vesicles from the media conditioned by the MSCs.

5. The method of claim 4, wherein the composition does not comprise the media conditioned by the MSCs.

6. The method of claim 1, wherein the MSCs are primary cells or a cell line.

7. The method claim 6, wherein the MSCs are from bone barrow, placenta, umbilical cord blood, adipose tissue, adult muscle, corneal stroma, or the dental pulp of deciduous teeth.

8. The method of claim 7, wherein the MSCs are adipose-derived stem cells.

9. The method of claim 1, wherein the extracellular vesicles comprise or consist of ectosomes, microvesicles (MV), microparticles, exosomes, oncosomes, apoptotic bodies (AB), tunneling nanotubes (TNT), or a combination thereof.

10. The method of claim 9, wherein the extracellular vesicles comprise or consist of exosomes, microvesicles or a combination thereof.

11. The method of claim 10, wherein the extracellular vesicles comprise or consist of a vesicles having a size of between about 20 nm and about 500 nm, or between about 20 nm and about 250 nm, or between about 20 nm and about 200 nm, or between about 20 nm and about 150 nm, or between about 20 nm and about 100 nm.

12. The method of claim 11, wherein the extracellular vesicles comprise CD9, CD36, or a combination thereof

13. The method of claim 11, wherein the extracellular vesicles comprise one or more of miR-199a-3p, miR-145-5p, miR-143-3p, miR-377-3p, miR-100-3p, miR-29a-3p, miR-495-3p, miR-29c-3p, miR-658, miR-493-3p, miR-184, and miR-27a-3p.

14. The method of claim 1 comprising increasing the proliferation, migration, and/or tube formation of lymphatic endothelial cells, increasing expression of one or more lymphatic markers (e.g., hyaluronan receptor-1(LYVE-1), vascular endothelial growth factor receptor-3 (VEGFR-3), prospero homeobox 1 (Prox1), and/or podoplanin) in lymphatic endothelial cells, increasing angiogenesis, increasing lymphangiogeneisis, reducing inflammatory response, decreasing fibrosis formation, enlarging circumference and/or inducing formation of capillary vessels and/or lymphatic vessels, inducing formation of vessels that express both vascular and lymphatic markers, increasing drainage routes (e.g., for accumulated fluids), increasing HIF1-alpha expression and/or activity, reducing Prohibitin (PHB) expression and/or activity, or a combination thereof in the subject.

15. The method of claim 1, wherein the subject has a blockage in the lymphatic system, optionally wherein the blockage prevents lymph fluid from draining well, and wherein the fluid buildup leads to swelling.

16. The method of claim 1, wherein the subject has one or more symptoms selected from swelling of part or all of the arm(s) and/or leg(s), a feeling of heaviness or tightness, restricted range of motion, aching or discomfort, recurring infections, and fibrosis in one or both arms and/or legs.

17. The method of claim 1, wherein the subject has been diagnosed with lymphedema.

18. The method of claim 1, wherein the composition is administered by local injection or infusion at or adjacent to a site of interest.

19. The method of claim 18, wherein the site of interest is in one or both arms and/or legs.

20. The method of claim 18, wherein the site of interest is a site of lymphatic blockage and/or lymphedema.


SEQUENCE LISTING (102 sequences)

SEQ ID NO: 1 - RNA, 22 nt, Homo sapiens
caaaaaucucaauuacuuuugc

SEQ ID NO: 2 - RNA, 22 nt, Homo sapiens
ugccugucuacacuugcugugc

SEQ ID NO: 3 - RNA, 23 nt, Homo sapiens
aaagugcugcgacauuugagcgu

SEQ ID NO: 4 - RNA, 22 nt, Homo sapiens
uggaauguaaggaagugugugg

SEQ ID NO: 5 - RNA, 21 nt, Homo sapiens
gaacggcuucauacaggaguu

SEQ ID NO: 6 - RNA, 23 nt, Homo sapiens
cccaguguucagacuaccuguuc

SEQ ID NO: 7 - RNA, 22 nt, Homo sapiens
acagcaggcacagacaggcagu

SEQ ID NO: 8 - RNA, 22 nt, Homo sapiens
acaguagucugcacauugguua

SEQ ID NO: 9 - RNA, 22 nt, Homo sapiens
gccugcugggguggaaccuggu

SEQ ID NO: 10 - RNA, 22 nt, Homo sapiens
aacccguagauccgaacuugug

SEQ ID NO: 11 - RNA, 23 nt, Homo sapiens
guccaguuuucccaggaaucccu

SEQ ID NO: 12 - RNA, 23 nt, Homo sapiens
cccaguguuuagacuaucuguuc

SEQ ID NO: 13 - RNA, 21 nt, Homo sapiens
ugagaugaagcacuguagcuc

SEQ ID NO: 14 - RNA, 21 nt, Homo sapiens
uacucaaaaagcugucaguca

SEQ ID NO: 15 - RNA, 22 nt, Homo sapiens
cugcgcaagcuacugccuugcu

SEQ ID NO: 16 - RNA, 22 nt, Homo sapiens
cuguacagccuccuagcuuucc

SEQ ID NO: 17 - RNA, 23 nt, Homo sapiens
uuauugcuuaagaauacgcguag

SEQ ID NO: 18 - RNA, 22 nt, Homo sapiens
cuccuauaugaugccuuucuuc

SEQ ID NO: 19 - RNA, 22 nt, Homo sapiens
ggauuccuggaaauacuguucu

SEQ ID NO: 20 - RNA, 22 nt, Homo sapiens
uuugguccccuucaaccagcua

SEQ ID NO: 21 - RNA, 22 nt, Homo sapiens
auaagacgaacaaaagguuugu

SEQ ID NO: 22 - RNA, 22 nt, Homo sapiens
uuguacaugguaggcuuucauu

SEQ ID NO: 23 - RNA, 22 nt, Homo sapiens
cuauacaaccuacugccuuccc

SEQ ID NO: 24 - RNA, 22 nt, Homo sapiens
ugagguaguaguuugugcuguu

SEQ ID NO: 25 - RNA, 22 nt, Homo sapiens
ggugcagugcugcaucucuggu

SEQ ID NO: 26 - RNA, 23 nt, Homo sapiens
uggugcggagagggcccacagug

SEQ ID NO: 27 - RNA, 22 nt, Homo sapiens
caucaucgucucaaaugagucu

SEQ ID NO: 28 - RNA, 22 nt, Homo sapiens
uuugguccccuucaaccagcug

SEQ ID NO: 29 - RNA, 22 nt, Homo sapiens
agagguugcccuuggugaauuc

SEQ ID NO: 30 - RNA, 22 nt, Homo sapiens
ugagguaguagguugugugguu

SEQ ID NO: 31 - RNA, 22 nt, Homo sapiens
aucacacaaaggcaacuuuugu

SEQ ID NO: 32 - RNA, 23 nt, Homo sapiens
acuccauuuguuuugaugaugga

SEQ ID NO: 33 - RNA, 22 nt, Homo sapiens
acggguuaggcucuugggagcu

SEQ ID NO: 34 - RNA, 22 nt, Homo sapiens
caagcuuguaucuauagguaug

SEQ ID NO: 35 - RNA, 22 nt, Homo sapiens
ucccugagacccuaacuuguga

SEQ ID NO: 36 - RNA, 22 nt, Homo sapiens
ucggauccgucugagcuuggcu

SEQ ID NO: 37 - RNA, 22 nt, Homo sapiens
acugauuucuuuugguguucag

SEQ ID NO: 38 - RNA, 22 nt, Homo sapiens
uauagggauuggagccguggcg

SEQ ID NO: 39 - RNA, 22 nt, Homo sapiens
ggagaaauuauccuuggugugu

SEQ ID NO: 40 - RNA, 21 nt, Homo sapiens
caaaacgugaggcgcugcuau

SEQ ID NO: 41 - RNA, 22 nt, Homo sapiens
agaucgaccguguuauauucgc

SEQ ID NO: 42 - RNA, 21 nt, Homo sapiens
aucauagaggaaaauccacgu

SEQ ID NO: 43 - RNA, 21 nt, Homo sapiens
aauauaacacagauggccugu

SEQ ID NO: 44 - RNA, 21 nt, Homo sapiens
aacauagaggaaauuccacgu

SEQ ID NO: 45 - RNA, 22 nt, Homo sapiens
uagguuauccguguugccuucg

SEQ ID NO: 46 - RNA, 22 nt, Homo sapiens
gaauguugcucggugaaccccu

SEQ ID NO: 47 - RNA, 22 nt, Homo sapiens
ugagaacugaauuccauggguu

SEQ ID NO: 48 - RNA, 22 nt, Homo sapiens
uagcaccaucugaaaucgguua

SEQ ID NO: 49 - RNA, 22 nt, Homo sapiens
cugaagcucagagggcucugau

SEQ ID NO: 50 - RNA, 22 nt, Homo sapiens
gaaguuguucgugguggauucg

SEQ ID NO: 51 - RNA, 23 nt, Homo sapiens
agguuacccgagcaacuuugcau

SEQ ID NO: 52 - RNA, 22 nt, Homo sapiens
cagcagcaauucauguuuugaa

SEQ ID NO: 53 - RNA, 22 nt, Homo sapiens
cuauacaaucuauugccuuccc

SEQ ID NO: 54 - RNA, 22 nt, Homo sapiens
gucauacacggcucuccucucu

SEQ ID NO: 55 - RNA, 21 nt, Homo sapiens
aauaauacaugguugaucuuu

SEQ ID NO: 56 - RNA, 22 nt, Homo sapiens
guagauucuccuucuaugagua

SEQ ID NO: 57 - RNA, 22 nt, Homo sapiens
uggaauguaaagaaguauguau

SEQ ID NO: 58 - RNA, 22 nt, Homo sapiens
cuauacgaccugcugccuuucu

SEQ ID NO: 59 - RNA, 21 nt, Homo sapiens
aaucauucacggacaacacuu

SEQ ID NO: 60 - RNA, 23 nt, Homo sapiens
ugucacucggcucggcccacuac

SEQ ID NO: 61 - RNA, 22 nt, Homo sapiens
aaucguacagggucauccacuu

SEQ ID NO: 62 - RNA, 21 nt, Homo sapiens
uaguagaccguauagcguacg

SEQ ID NO: 63 - RNA, 22 nt, Homo sapiens
auucugcauuuuuagcaaguuc

SEQ ID NO: 64 - RNA, 21 nt, Homo sapiens
uuaauaucggacaaccauugu

SEQ ID NO: 65 - RNA, 22 nt, Homo sapiens
aacacaccugguuaaccucuuu

SEQ ID NO: 66 - RNA, 23 nt, Homo sapiens
uagcagcgggaacaguucugcag

SEQ ID NO: 67 - RNA, 22 nt, Homo sapiens
cugggagaggguuguuuacucc

SEQ ID NO: 68 - RNA, 22 nt, Homo sapiens
aguucuucaguggcaagcuuua

SEQ ID NO: 69 - RNA, 25 nt, Homo sapiens
ggcggagggaaguagguccguuggu

SEQ ID NO: 70 - RNA, 21 nt, Homo sapiens
agaggauacccuuuguauguu

SEQ ID NO: 71 - RNA, 22 nt, Homo sapiens
aaacaaacauggugcacuucuu

SEQ ID NO: 72 - RNA, 23 nt, Homo sapiens
ucuuggaguaggucauugggugg

SEQ ID NO: 73 - RNA, 21 nt, Homo sapiens
ugguagacuauggaacguagg

SEQ ID NO: 74 - RNA, 22 nt, Homo sapiens
aagcugccaguugaagaacugu

SEQ ID NO: 75 - RNA, 22 nt, Homo sapiens
uaugucugcugaccaucaccuu

SEQ ID NO: 76 - RNA, 22 nt, Homo sapiens
agggacuuucaggggcagcugu

SEQ ID NO: 77 - RNA, 22 nt, Homo sapiens
ugaaggucuacugugugccagg

SEQ ID NO: 78 - RNA, 22 nt, Homo sapiens
ugugacugguugaccagagggg

SEQ ID NO: 79 - RNA, 22 nt, Homo sapiens
aaucauacacgguugaccuauu

SEQ ID NO: 80 - RNA, 22 nt, Homo sapiens
uauguaacacgguccacuaacc

SEQ ID NO: 81 - RNA, 22 nt, Homo sapiens
uuuuucauuauugcuccugacc

SEQ ID NO: 82 - RNA, 22 nt, Homo sapiens
aaacauucgcggugcacuucuu

SEQ ID NO: 83 - RNA, 22 nt, Homo sapiens
uauacaagggcaagcucucugu

SEQ ID NO: 84 - RNA, 22 nt, Homo sapiens
cacgcucaugcacacacccaca

SEQ ID NO: 85 - RNA, 22 nt, Homo sapiens
uauguaacaugguccacuaacu

SEQ ID NO: 86 - RNA, 22 nt, Homo sapiens
uagcuuaucagacugauguuga

SEQ ID NO: 87 - RNA, 22 nt, Homo sapiens
uggugggccgcagaacaugugc

SEQ ID NO: 88 - RNA, 23 nt, Homo sapiens
uaaaucccauggugccuucuccu

SEQ ID NO: 89 - RNA, 22 nt, Homo sapiens
uagcaccauuugaaaucgguua

SEQ ID NO: 90 - RNA, 22 nt, Homo sapiens
uggacggagaacugauaagggu

SEQ ID NO: 91 - RNA, 22 nt, Homo sapiens
ugggucuuugcgggcgagauga

SEQ ID NO: 92 - RNA, 25 nt, Homo sapiens
aaaggauucugcugucggucccacu

SEQ ID NO: 93 - RNA, 21 nt, Homo sapiens
uucacaguggcuaaguuccgc

SEQ ID NO: 94 - RNA, 22 nt, Homo sapiens
ugaaacauacacgggaaaccuc

SEQ ID NO: 95 - RNA, 22 nt, Homo sapiens
aucauagaggaaaauccauguu

SEQ ID NO: 96 - RNA, 22 nt, Homo sapiens
ggauaucaucauauacuguaag

SEQ ID NO: 97 - RNA, 26 nt, Homo sapiens
aaguaguugguuuguaugagaugguu

SEQ ID NO: 98 - RNA, 21 nt, Homo sapiens
cacauuacacggucgaccucu

SEQ ID NO: 99 - RNA, 23 nt, Homo sapiens
ucaagagcaauaacgaaaaaugu

SEQ ID NO: 100 - RNA, 21 nt, Homo sapiens
caacaccagucgaugggcugu

SEQ ID NO: 101 - RNA, 22 nt, Homo sapiens
agagguaguagguugcauaguu

SEQ ID NO: 102 - RNA, 22 nt, Homo sapiens
aaucauacagggacauccaguu